(19) United States
(12) Patent Application Publication
(10) Pub. No.: US 2013/0330728 A1
(43) Pub. Date: Dec. 12, 2013

(54) MICROBIAL SIGNATURES AS INDICATORS OF RADIATION EXPOSURE

(71) Applicant: John Edward Baker, Wauwatosa, WI (US)

(72) Inventor: John Edward Baker, Wauwatosa, WI (US)

(21) Appl. No.: 13/499,256

(22) PCT Filed: Feb. 24, 2012
(86) PCT No.: PCT/US12/26541

§ 371 Date: Jun. 29, 2012

Related U.S. Application Data
(60) Provisional application No. 61/446,696, filed on Feb. 25, 2011.
(60) Provisional application No. 61/451,930, filed on Mar. 11, 2011.
(60) Provisional application No. 61/479,786, filed on Apr. 27, 2011.
(60) Provisional application No. 61/497,452, filed on Jun. 15, 2011.

(51) Int. Cl. C12Q 1/68

(52) U.S. Cl. CPC C12Q 1/68
USPC 435/6.12

(57) ABSTRACT

The present disclosure defines a method for identifying and characterizing radiation exposure after, for example, a nuclear event such as detonation of a nuclear weapon or a nuclear meltdown of a nuclear power plant, using changes in microbiomes. The present disclosure demonstrates that microbiomes are reproducibly and detectably altered by exposure to radiation. As described herein, microbial signatures can be used to characterize components of microbiomes that are altered by exposure to radiation.

### RELATED APPLICATIONS

This application claims priority to U.S. Provisional Patent Application Ser. Nos. 61/446,696, filed Feb. 25, 2011; 61/451,930, filed Mar. 11, 2011; 61/479,786, filed Apr. 27, 2011; and 61/491,452, filed May 31, 2011; the entirety of each of which is hereby incorporated by reference.

### GOVERNMENT SUPPORT

The United States Government has provided grant support utilized in the development of the present invention. In particular, National Institutes of Health grant number AI080363 has supported development of this invention. The United States Government may have certain rights in the invention.

### BACKGROUND

As of 2011, it's estimated that there are more than 20,500 nuclear warheads worldwide, with around 4,800 kept on standby for potential use. Even a small nuclear weapon can devastate an entire city. In addition, there are 439 operational nuclear reactors worldwide; a malfunction at a nuclear power plant has potential to do significant damage to surrounding areas.

### SUMMARY

The present invention encompasses the recognition that reproducible and detectable changes occur in microbiome composition and/or activity in response to radiation exposure. The present invention permits identification and/or characterization of microbial signatures reflecting such changes, and also provides systems for using such microbial signatures, for example to assess or detect extent and/or type of radiation to which an individual or area may have been exposed.

In some embodiments, a microbial signature comprises a level or levels of one or more microbes or components or products thereof and is sufficient to distinguish or characterize a microbiome exposed to radiation (and/or to a particular extent or type of radiation) relative to a microbiome that has not been so exposed (e.g., has not been exposed at all, or has been exposed to a different extent and/or type), or has been exposed to a known reference dose and/or type of radiation. For example, in some embodiments, microbial signatures obtained from gastrointestinal microbiomes of individuals suspected of or suffering from radiation exposure are sufficient to diagnose individuals when compared with microbial signatures of gastrointestinal microbiomes of unexposed individuals and/or of reference exposed individuals.

In accordance with the present invention, microbial signatures are defined for particular microbiota samples relative to appropriate reference microbiota samples. In some embodiments, particular microbiota samples share a common feature of radiation exposure that is not shared by reference microbiota samples. In some embodiments particular microbiota samples differ from reference microbiota samples in that they are samples of a different source. In some embodiments the particular microbiota samples differ from reference microbiota samples in that the microbiota reference samples are historical microbiota samples of the same or a different source.

In certain embodiments, the present disclosure provides methods for identifying and/or characterizing exposure to radiation comprising providing a reference microbial signature that correlates with extent and/or type of exposure to radiation and determining a microbial signature present in a microbiota sample from an individual whose exposure to radiation is to be identified or characterized. In some embodiments, a microbiota sample comprises a sample of one or more types of microbes found in a gastrointestinal tract of a subject. In some embodiments, the microbial signature comprises a level or set of levels of one or more 16S rRNA gene sequences of one or more types of microbes.

In certain embodiments, the present disclosure provides methods for defining a microbial signature that correlates with an aspect of radiation exposure. For example, in some embodiments, the present disclosure provides methods comprising steps of determining a first set of levels of one or more types of microbes, or components or products thereof, in a first collection of microbiota samples, where each sample in the first collection of microbiota samples shares a common feature of radiation exposure, determining a second set of levels of the one or more types of microbes or components or products thereof in a second collection of microbiota samples, which second collection of microbiota samples does not share the common feature of radiation exposure but is otherwise comparable to the first set of microbiota samples, and identifying a microbial signature comprising levels within the first or second set that correlates with presence or absence of the common feature of radiation exposure. In some embodiments, a common feature of radiation exposure comprises an intensity of exposure ranging from 0 to 10 Grays (Gy). In some embodiments, a set of levels of one or more types of microbes or components or products thereof comprises a set of levels of 16S rRNA gene sequences of one or more types of microbes found in a gastrointestinal tract from which microbiota samples are collected

### BRIEF DESCRIPTION OF THE DRAWING

FIG. 1 shows a scatter plot of data from 6 rats to show data variance amongst irradiated rats. Data shown herein was used to generate data shown in FIGS. 7A and 7B. Approximately 5% of the 432 values are missing due to a rat not being able to produce feces at time of sampling. Each data point consist of at least 4 biological replicates.

FIG. 2 presents a bar chart showing proportions of Operational Taxonomical Units (OTUs) present in rat feces classified at family level. For each sample, the 6 richest members of family rank are shown. Each color block represents a percentage of OTUs detected within a family compared to total number of OTUs detected within the 6 richest families.

FIGS. 3A-3B illustrate intestinal microbial community analysis in feces pre- and post-irradiation. In FIG. 3A, differences in composition of 16S rRNA sequences measured by PhyloChip are used to calculate the Bray-Curtis distance between rat feces samples. Presence-absence scoring for each hybridizing signal in all 7484 OTUs was incorporated in the analysis. Non-metric multidimensional scaling ordination of samples showed microbial communities were significantly different by day (p<0.001) but not by rat (p<0.09), as determined by the Adonis test, and delineated with lines for clarity. FIG. 3B demonstrates hierarchical clustering showing phylogenetic relationships of microbiota in rat feces. Samples were clustered using the farthest neighbor distance (complete linkage) algorithm to show strong dependence of microbiota on day post irradiation.

FIGS. 4A-4B illustrate candidate biomarkers for radiation exposure.

FIG. 4A shows a Venn diagram illustrating abundance of OTUs exhibiting statistically significant changes between background, day 0, and day 11 (Day 11); background and day 21 (Day 21); and background and combined days 4-21 (All Days). Numbers in black indicate number of OTUs that are shared between each analysis. Nonmetric multidimensional scaling ordination of samples based on the 147 common OTUs found in FIG. 4A shown in FIG. 4B showed distance separation by day (p<0.001) but not by rat (p<0.09). Data points are delineated with lines for clarity.

FIG. 5 presents heatmaps highlighting trends of OTUs that increase (blue) and decrease (red) following irradiation. Log₂ fold changes of day 0 compared with an average of days 4, 11, and 21 are shown along with t-test p-values.

FIG. 6 presents a line graph showing persistent changes in specific OTU abundance following radiation exposure. Abundance of three representative OTUs: 31902, 42924, and 39153 showing increased, stable, and decreased 16S rRNA expression is shown. Error bars represent within group variation for 5 rats at each time point. Ratio of 39102 abundance relative to 39153, i.e. log₂ 39102-log₂ 39153 served as a potential composite biomarker for irradiation. Values of this biomarker are −3.95, 0.98, 1.60, and 1.42 at days 0, 4, 11, and 21 respectively. Values were calculated independently for each day and are statistically significant as compared to day 0 (*=p<0.001).

FIGS. 7A-7B illustrate transient or no changes in multiple individual human bacteria present in feces of rats exposed to 10 (FIG. 7A) and 18 (FIG. 7B) Gy irradiation. Abundance of Proteobacteria was increased by almost 1000 fold following irradiation while Clostridia and Bacteroidetes abundances were relatively stable. A 10 fold drop in Clostridia was observed only in feces of 18 Gy irradiated rats. Data are mean±standard deviation. n=5/group (*=p<0 05 vs Day 0).

FIG. 8 presents a collection of line graphs illustrating abundance of biomarkers in feces of rats exposed to 10 and 18 Gy irradiation at 0, 2, 4, 8, 11, 15, and 21 days post exposure.

FIGS. 9A-9B demonstrate PCR confirmation of biomarker dynamics in feces of rats exposed to 10 (FIG. 9A) and 18 (FIG. 9B) Gy irradiation. Dashed lines show the ratio of “acutely increased/decreased” biomarkers and solid lines show the ratio of “chronically increased/decreased” biomarkers. Data are mean±standard deviation. n=5/group (*=p<0.05 vs Day 0).

FIGS. 10A-10B show bar graphs illustrating the stability of bacterial populations across age (FIG. 10A) strain and diet (FIG. 10B) in rats not exposed to radiation.

FIG. 11 presents a chart mapping rat biomarker OTUs to human microbiome project pyrosequencing data.

FIG. 12 shows a bar graph illustrating abundance of different microbe types in rats treated with different antibiotics. Orally administered vancomycin and a mixture of streptomycin, bacitracin polymyxin B and neomycin alter abundance of intestinal microbiota present in rat feces.

### DEFINITIONS

Antibiotic: As used herein, the term “antibiotic agent” means any of a group of chemical substances, isolated from natural sources or derived from antibiotic agents isolated from natural sources, having a capacity to inhibit growth of, or to destroy bacteria, and other microorganisms, used chiefly in treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, sulfonamides, fluoroquinolones, and lincosamides.

Antibacterial agents also include antibacterial peptides. Examples include but are not limited to maximum H5, dermcidin, cecropins, andropin, moricin, ceratotoxin, melittin, magainin, dermaseptin, bombinin, brevinin-1, esculentins, buforin II, CAP18, LL37, abaecin, apidaecins, prophenin, indolicidin, brevinins, protegrin, tachyplesins, defensins, and or drosomycin.

Comparable: Sufficiently similar to permit comparison, but differing in at least one feature.

Correlates: The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Differentiates: The term “differentiates”, as used herein, indicates defining or distinguishing from other entities (e.g., comparable entities). In some embodiments, differentiates means distinguishing from other types with which present together in source and/or sample.

Microbe: The term “microbe” is typically used in the art to refer to a microscopically small organisms such as a bacterium, fungus, protozoan, or virus. In some embodiments, a microbe is a bacterium, archaeon, unicellular fungus (e.g., yeast), alga, or a protozoa (e.g., plasmodia as a malaria pathogen). In some embodiments, microbes are characterized according to their kingdom. In some embodiments, microbes are characterized according to their phylum. In some embodiments, microbes are characterized according to their class. In some embodiments, microbes are characterized according to their family. In some embodiments, microbes are characterized according to their genus. In some embodiments, microbes are characterized according to their species. In some embodiments, microbes are characterized according to their subspecies. In some embodiments, microbes are characterized according to their strain. Occasionally additional taxonomic class(es), e.g., serovars or serotypes, are used for differentiating microbes, such as bacteria, included within a subspecies. Serovars and serotypes are distinguished by their different types of attachment behavior at a cell membrane. In some embodiments, genus and species are utilized to identify and/or characterize a microbe (e.g., in a sample). In some embodiments, subspecies, serotype and/or strain are utilized to identify and/or characterize a microbe (e.g., in a sample). Alternatively or additionally, in some embodiments, a microbe (e.g., in a sample) is identified and/or characterized using one or more distinguishing characteristics such as pathogenicity (i.e., an ability to bring on a particular illness), or resistance to one or more antibiotics, metabolic profiles, morphology, etc.

Microbial Types: As will be understood from the context, the term “microbial types” or “types of microbes” is used herein to indicate a grouping of microbes with a common feature. In some embodiments, a microbial type is a group of microbes sharing a common detectable feature. In some embodiments, a common detectable feature is or comprises presence or amount of a particular DNA sequence. In some embodiments, a common detectable feature is or comprises presence or amount of a particular RNA transcript. In some embodiments, a common detectable feature is or comprises presence or amount of a polypeptide (e.g., a microbially-produced polypeptide). In some embodiments, a common detectable feature is or comprises presence or level of an enzymatic activity (e.g., of a microbial enzyme). In some embodiments, microbes of a common type are microbes of a particular classification, according to standard taxonomy. Those of skill in the art will understand that the term “microbial type” as used herein is not restricted to a specific degree of resolution; different features may be detected using technologies that achieve different levels of resolution. In some embodiments, microbes of a common type are microbes of the same microbial kingdom. In some embodiments, microbes of a common type are microbes of the same microbial phylum. In some embodiments, microbes of a common type are microbes of the same microbial class. In some embodiments, microbes of a common type are microbes of the same microbial family. In some embodiments, microbes of a common type are microbes of the same microbial genus. In some embodiments, microbes of a common type are microbes of the same microbial species. In some embodiments, microbes of a common type are microbes of the same microbial subspecies. In some embodiments, microbes of a common type are microbes of the same microbial serovar. In some embodiments microbes of a common type are microbes of the same microbial serotype. In some embodiments, microbes of a common type are microbes of the same strain.

Radiation: As will be understood from context, the term “radiation” can refer to any type of emission of energy as electromagnetic waves or as moving subatomic particles. In some embodiments in accordance with the present invention, radiation comprises ionizing radiation. Ionizing radiation is radiation of sufficiently high energy to ionize atoms. Types of ionizing radiation include but are not limited to alpha radiation, beta radiation, cosmic radiation, neutron radiation, X-ray radiation, and gamma radiation. In some embodiments, radiation comprises non-ionizing radiation. Types of non-ionizing radiation include but are not limited to visible light, infrared light, microwave radiation, radiowaves, very low frequency radiation, extremely low frequency radiation, thermal radiation, and black body radiation.

Reference: As will be understood from context, a reference sample or individual is one that is sufficiently similar to a particular sample or individual of interest to permit a relevant comparison. In some embodiments, information about a reference sample is obtained simultaneously with information about a particular sample. In some embodiments, information about a reference sample is historical. In some embodiments, information about a reference sample is stored for example in a computer-readable medium. In some embodiments, comparison of a particular sample of interest with a reference sample establishes identity with, similarity to, or difference of the particular sample of interest relative to the reference.

Sample: As used herein, the term “sample” refers to a biological or environmental sample obtained from a source of interest. In some embodiments, a source of interest comprises an organism, such as an insect, animal, human, or plant; in some embodiments, a source of interest comprises soil, sediment, ground water, surface water and/or air from a geographic location. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from the individual from whom the sample is obtained. In some embodiments, obtained cells are or include microbial cells of the individual's microbiome. In some embodiments, a sample is a “primary sample” obtained directly from a source of interest by any appropriate means. For example, in some embodiments, a primary biological sample is obtained by a method selected from the group consisting of biopsy (e.g., fine needle aspiration or tissue biopsy), surgery, collection of body fluid (e.g., blood, lymph, feces etc.), etc. In some embodiments, a primary environmental sample is obtained by digging, core sampling, and/or extracting or combinations thereof. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Substantially: As used herein, the term “substantially” refers to a qualitative condition of exhibiting total or near-total extent or degree of a characteristic or property of interest. Those of ordinary skill in the biological arts will appreciate that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Transcript: As used herein, the term “transcript” refers to a molecule as transcribed or alternately as processed in one or more steps of splicing, ect.

### DETAILED DESCRIPTION OF CERTAIN EMBODIMENTS

### Radiation Exposure

In recent history, various nuclear events have devastated local populations. Events like the Chernobyl nuclear power plant disaster in 1986, the atomic bombs dropped on Hiroshima and Nagasaki, Japan during World War II, and more recently the Fukushima Nuclear Power Plant disaster, result in large numbers of casualties and require rapid medical screening and treatment of large numbers of exposed survivors in areas where access to medical care is hindered by damage to infrastructures and mass hysteria.

Methods in accordance with the present invention provide a means for identifying and/or characterizing exposure to radiation. During a nuclear event, fast and reliable means are needed to identify radiation-exposed individuals and characterize their exposure. Humans are highly sensitive to radiation exposure, but appropriate medical treatment can have a dramatic impact on chances of survival and/or extent of disease or suffering. In certain situations, it may be critical to not only identify, but also to quantify radiation dose because appropriate medical treatment can be highly dose dependent.

After such a nuclear event, fast and reliable means for identifying radiation-exposed individuals can provide a means for excluding unexposed individuals from treatment and/or for identifying extent or type of treatment appropriate to exposed individuals. Because exposed individuals may be initially asymptomatic and because radiation is undetectable by human senses, large numbers of potentially exposed individuals requiring screening for exposure will often vastly outnumber individuals requiring treatment, especially if a nuclear event occurs in a populated area. Having capabilities to exclude those who have not been exposed from treatment provides vital information, useful not only for managing available medical resources, but also for preventing widespread panic within a population.

Examples of sources of radiation exposure include but are not limited to nuclear power plants, nuclear weapons, cosmic rays, radiation therapy, nuclear materials, radiopharmaceuticals, X-ray tubes, particle accelerators, exposure to radon-222, exposure to thorium-232, exposure to uranium-235 and -238, exposure to potassium-40, exposure to radium-226, smoke detectors, airport luggage screeners, radiation diagnostics (CT scans), radiologic dirty bombs and space travel or any combination thereof.

Without appropriate medical care, humans have a median lethal dose of radiation of (LD50/60—a dose that kills 50% of an exposed population within 60 days after exposure) 4.5 Gy (Mole, R. H. “The LD50 for uniform low LET irradiation of man” Br J. Radiol. 57:355-69, 1984). However, with appropriate medical treatment, this dose can be doubled. Appropriate medical treatment is highly dose dependent. Doses under 1 Gy generally do not require treatment. Doses from 1 to 7 Gy are generally treated with antibiotics, platelets, or cytokine treatment or any combination thereof. Appropriate cytokines for treatment include but are not limited to granulocyte colony-stimulating factor, filgrastim, pegylated granulocyte colony-stimulating factor, pegfilgrastim, granulocyte macrophage colony-stimulating factor, and/or sargramostim. Doses from 7 to 10 Gy are treated with bone marrow transplantation. Doses over 10 Gy are generally believed to result in lethal gastrointestinal damage.

In some embodiments of the present invention, radiation exposure, or exposure to radiation, comprises any amount of radiation to which an individual or object has been exposed. In some embodiments, radiation exposure comprises exposure to non-ionizing radiation. In some embodiments, radiation exposure comprises exposure to ionizing radiation. In some embodiments, radiation exposure comprises exposure to between 0 and 1 Gy of ionizing radiation. In some embodiments, radiation exposure comprises exposure to 1, 2, 3, 4, 5, 6, 7, 8, 9, 10 or more Grays of ionizing radiation. A Gray is a measure of radiation exposure defined as absorption of one joule of ionizing radiation by one kilogram of matter.

Clinical manifestations of radiation exposure include but are not limited to loss of and/or damage to bone marrow cells, decreased lymphocytes, altered levels of granulocytes, gastrointestinal symptoms including loss of intestinal crypts and gastrointestinal barrier breakdown, loss of and/or damage to epidermal and/or dermal cells and combinations thereof.

Affected individuals may immediately show symptoms of radiation exposure. Affected individuals may be initially asymptomatic and then begin to show symptoms of exposure after a period of time. Affected individuals may begin to show symptoms after 1, 2, 3, 4, 5, 6, 7, 8, 9, 10 or more seconds. Affected individuals may begin to show symptoms after 1, 2, 3, 4, 5, 6, 7, 8, 9, 10 or more minutes. Affected individuals may begin to show symptoms after 1, 2, 3, 4, 5, 6, 7, 8, 9, 10 or more hours. Affected individuals may begin to show symptoms after 1, 2, 3, 4, 5, 6, 7, 8, 9, 10 or more days. Affected individuals may be asymptomatic.

In some embodiments, symptoms of radiation exposure include but are not limited to nasal bleeding, mouth bleeding, gum bleeding, rectum bleeding, bloody stool, bruising, confusion, dehydration, diarrhea, fainting, fatigue, fever, hair loss, inflammation of exposed areas (redness, tenderness, swelling, bleeding), mouth ulcers, nausea and vomiting, open sores on the skin, skin burns (redness, blistering), sloughing of skin, esophageal ulcers, stomach ulcers, intestinal ulcers, vomiting blood, weakness and combinations thereof.

Current methods of assessing radiation exposure include but are not limited to assessment of symptoms present, obtaining biological samples for radiological monitoring, determination of absolute lymphocyte counts, lymphocyte depletion kinetics, chromosome-aberration cytogenetic assays, assaying eukaryotic gene and protein expression in blood, assaying eukaryotic gene and protein expression in urine, and electron spin resonance of dental enamel and nail clippings.

Monitoring lymphocyte depletion kinetics is generally believed to be a practical method to assess radiation dose within hours or days following a radiation exposure. Lymphocyte depletion kinetics is able to detect doses of 1-10 Gy with a resolution of 2 Gy. However, assaying lymphocyte depletion kinetics requires hematology laboratory capabilities, and a minimum of 3 complete blood counts over four days immediately following radiation exposure. For more accurate results, ideally 6 complete blood counts are needed within 2-3 days of exposure with a first blood count obtained within 4 hours of exposure.

Because monitoring lymphocyte depletion kinetics would likely be difficult following a major nuclear event, chromosome-aberration cytogenetic assays remain the gold standard for quantifying radiation exposure following a major nuclear event. However, a major disadvantage of this assay is that results are not available for several days. Blood samples cannot be taken until 24 hours after exposure and then take between 48 and 72 hours to process. Clearly a need exists for a means of quantifying radiation exposure easily and rapidly following a nuclear event.

### Microbiome

A human body typically contains ten times as many microbial (and particularly bacterial) cells as it has human cells. Many or most of such microbes are harmless, or even beneficial, to their human host. Increasingly, research demonstrates that such microbes play a significant role in maintaining and/or promoting human health. Gastrointestinal bacteria are a well studied example. These bacteria are thought to provide a variety of important functions including but not limited to aiding in carbohydrate digestion, regulating of intestinal cell growth, repressing pathogenic microbial growth, promoting development of intestinal mucosal immunity, metabolizing carcinogens, and preventing allergies and inflammatory bowel diseases.

Most other multicellular organisms similarly exist in commensal relationships with large amounts of microbes. Examples of symbiotic relationships between microbes and hosts are prevalent amongst animals, plants and insects. The Euprymna scolopes squid has an organ for housing the luminescent bacteria Vibrio fischei, allowing the squid to feed at night. Plants of the legume family have nodules on their roots that house nitrogen fixing bacteria. Termite guts contain microbes that are able to digest cellulose.

All types and abundances of microbes in a particular environment comprise a microbiome. As microbes are nearly ubiquitous, microbiomes exist in most locations. In some embodiments a microbiome comprises microbes associated with any defined location. In some embodiments a microbiome comprises microbes associated with a non-living component of a natural environment. Examples include but are not limited rocks, soil, and water in any form, including water in natural bodies of water, puddles, pools, or droplets. In some embodiments a microbiome comprises microbes associated with a non-living component of a manufactured environment. Examples include but are not limited to a surface of a computer keyboard or mouse, a surface of manufacturing equipment, or a door handle. In some embodiments a microbiome comprises microbes associated with a living organism, or a particular portion, organ, tissue, or component thereof. In some embodiments, such an organism is a non-human multicellular organism that shares an environment with humans. In some embodiments, such an organism is a plant. In some embodiments, such an organism is an insect. In some embodiments, such an organism is an animal. In some embodiments, an animal is a mouse, rat, bird, cat, dog, wolf, coyote, deer, fox, skunk, rabbit, chipmunk, squirrel, horse, cow, goat, sheep, pig, possum, and cockroach. In some embodiments, an animal is a non-human primate. In some embodiments, an organism is a human.

Content (e.g., type and/or abundance of microbes present) and/or behavior (e.g., production of one or more markers, rate of respiration and/or proliferation, extent of migration, etc) of a microbiome can be shaped by local environments; in some embodiments; a single organism contains multiple different microbiomes, for example in different locations within or portions of their bodies. The human microbiome project (http://commonfund.nih.gov/hmp/) is characterizing the microbial communities found at several different sites on the human body, including nasal passages, oral cavities, skin, gastrointestinal tract, and urogenital tract. In some embodiments, a microbiome for use in accordance with the present invention is one associated with a particular site or location (e.g., tissue or organ) of an organism body. In some embodiments a microbiome comprises microbes associated with skin. In some embodiments a microbiome comprises microbes associated with teeth. In some embodiments a microbiome comprises microbes associated with oral mucosa. In some embodiments a microbiome comprises microbes associated with nasal passages. In some embodiments a microbiome comprises microbes associated with a urogenital system. In some embodiments a microbiome comprises microbes associated with a gastrointestinal tract.

In some embodiments, a microbiome comprises a single microbe. In some embodiments a microbiome comprises between 1 and a trillion or more individual microbes. In some embodiments, a microbiome comprises a single type of microbe. In some embodiments, a microbiome comprises between 1 and a million or more types of microbes. In some embodiments, a microbiome comprises between 500 and 5, 000 types of microbes. In some embodiments, a microbiome comprises between 1000 and 2, 000 types of microbes. Types of microbes that reside in the intestines are generally described at the phylum, class, order and family levels. In some embodiments, there are between 1000-1500 types of bacteria in gastrointestinal tract microbiomes.

### Microbiome Changes

The present invention teaches that microbiome composition and/or activity, and more particularly that changes in microbiome composition and/or activity can be informative about particular environmental conditions. The invention presented herein encompasses the finding that microbiome composition and/or activity can change in detectable and reproducible ways that are correlated with exposure to radiation.

In some embodiments, a change in microbiome composition and/or activity comprises any change in abundance and/or type of one or more types of microbes in a microbiome, and/or of one of more components produced thereby. In some embodiment a change in microbiome composition and/or activity comprises an increase in abundance of one or more types of microbes in a microbiome, or of one or more components produced thereby. Alternatively or additionally, in some embodiments, a change in microbiome composition and/or activity comprises a decrease in abundance of one or more types of microbes in a microbiome, and/or of one or more components produced thereby. In some embodiments, a change in microbiome composition and/or activity comprises an increase in abundance of one or more types of microbes, and/or of component(s) produced thereby, and also a decrease in abundance of one or more types of microbes in a microbiome, and/or of component(s) produced thereby.

In accordance with the present invention, microbiome changes that correlate with extent and/or type of radiation exposure are identified, characterized, and/or detected. In some embodiments, analysis of such changes involves controlling for and/or subtracting out effects of one or more other alterations in microbiome composition and/or activity.

Microbiome composition and/or activity can be detectably altered by events external or internal to a host organism. For example, oral ingestion of antibiotics by individuals can dramatically alter composition and/or activity of their gastrointestinal microbiomes.

In some embodiments a change in microbiome composition and/or activity occurs in response to disease in a host organism. In some embodiments a change in microbiome composition and/or activity occurs in response to infection of a host organism with pathogenic bacteria. In some embodiments a change in microbiome composition and/or activity occurs in response to a change in diet of a host organism. In some embodiments a change in microbiome composition and/or activity occurs in response to a change in water source of a host organism. In some embodiments a change in microbiome composition and/or activity occurs in response to a change in environment of a host organism, for example a person may move to a new city or country. In some embodiments a change in microbiome composition and/or activity occurs in response to a change in personal hygiene habits of a host organism. In some embodiments a change in microbiome composition and/or activity occurs in response to a change in weight of a host organism. In some embodiments a change in microbiome composition and/or activity occurs in response to a change in age of a host organism. In some embodiments a change in microbiome composition and/or activity occurs in response to a change in chemical exposure of a host organism.

In some embodiments a change in microbiome composition and/or activity occurs in response to exposure to microbiome altering agents. In some embodiments, microbiome altering agents comprise chemicals. In some embodiments, microbiome altering agents comprise antimicrobials. In some embodiments, microbiome altering agents comprise antibiotics. In some embodiments, microbiome altering agents comprise bacteria. In some embodiments, microbiome altering agents comprise probiotic bacteria. In some embodiments, microbe altering agents comprise antimicrobial peptides. In some embodiments, microbe altering agents comprise anti-fungals. In some embodiments, microbe altering agents comprise bacteriophages.

### Microbial Signature

The present invention encompasses the recognition that microbial signatures can be relied upon as proxy for microbiome composition and/or activity. Microbial signatures comprise data points that are indicators of microbiome composition and/or activity. Thus, according to the present invention, changes in microbiomes can be detected and/or analyzed through detection of one or more features of microbial signatures.

In some embodiments, a microbial signature includes information relating to absolute amount of one or more types of microbes, and/or products thereof. In some embodiments, a microbial signature includes information relating to relative amounts of one or more types of microbes and/or products thereof.

In some embodiments, a microbial signature includes information relating to presence, level, and/or activity of at least one type of microbes. In some embodiments, a microbial signature includes information relating to presence, level, and/or activity of between one and 10 types of microbes. In some embodiments, a microbial signature includes information relating to presence, level, and/or activity of between one and 100 types of microbes. In some embodiments, a microbial signature includes information relating to presence, level, and/or activity of between one and 1000 or more types of microbes. In some embodiments, a microbial signature includes information relating to presence, level, and/or activity of substantially all types of microbes within a microbiome.

In some embodiments, a microbial signature comprises a level or set of levels of one or more types of microbes or components or products thereof. In some embodiments, a microbial signature comprises a level or set of levels of one or more DNA sequences. In some embodiments, a microbial signature comprises a level or set of levels of one or more 16S rRNA gene sequences. In some embodiments, a microbial signature comprises a level or set of levels of 18S rRNA gene sequences. In some embodiments, a microbial signature comprises a level or set of levels of one or more RNA transcripts. In some embodiments, a microbial signature comprises a level or set of levels of one or more proteins. In some embodiments, a microbial signature comprises a level or set of levels of one or more metabolites.

16S and 18S rRNA gene sequences encode small subunit components of prokaryotic and eukaryotic ribsosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In methods in accordance with the present invention, a microbial signature is obtained and/or determined using a microbiota sample. A microbiota sample comprises a sample of microbes and or components or products thereof from a microbiome.

In some embodiments, a microbiota sample is collected by any means that allows recovery of microbes or components or products thereof of a microbiome and is appropriate to the relevant microbiome source. For example, where the microbiota sample of the gastrointestinal tract is obtained from a fecal sample.

### Quantifying Microbial Levels

In methods in accordance with the present invention, a microbial signature is obtained and/or determined by quantifying microbial levels. Methods of quantifying levels of microbes of various types are described herein.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

In some embodiments, a microbiota sample is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QJAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences. In some embodiments, 16S rRNA gene sequences are amplified using primer sequences listed in Table 1 or 2.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes or components or products thereof comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, determining a level or set of levels of one or more types of microbes or components or products thereof comprises determining a level or set of levels of one or more microbial proteins. Methods of quantifying protein levels are well known in the art and include but are not limited to western analysis and mass spectrometry. These and all other basic protein detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, determining a level or set of levels of one or more types of microbes or components or products thereof comprises determining a level or set of levels of one or more microbial metabolites. In some embodiments, levels of metabolites are determined by mass spectrometry. In some embodiments, levels of metabolites are determined by nuclear magnetic resonance spectroscopy. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

Microbial Signatures that Correlate with Radiation Exposure

The present invention encompasses the recognition that changes in microbial signature can be relied upon as proxy for changes in microbiome composition and/or activity. Thus, specific changes in a microbiome to be detected and/or analyzed will contribute to features of a microbial signature. In certain embodiments, the present invention is drawn to a method for defining a microbial signature indicative of radiation exposure by identifying those components of the microbiome that are affected by radiation exposure.

In some embodiments, defining a microbial signature that correlates with a feature of radiation exposure comprises any method that allows identification of types of microbes or components or products thereof that differ between exposed and non-exposed and/or that define or classify exposed microbiomes. In some embodiments, defining a microbial signature that correlates with an aspect of radiation exposure comprises determining a first set of levels of one or more types of microbes or components or products thereof in a first collection of microbiota samples, where each microbiota sample in the first collection of microbiota samples shares a common feature of radiation exposure; determining a second set of levels of the one or more types of microbes or components or products thereof in a second collection of microbiota samples, which second collection of microbiota samples does not share the common feature of radiation exposure but is otherwise comparable to the first set of microbiota samples; and identifying a microbial signature comprising levels within the first or second set that correlates with presence or absence of the common feature of radiation exposure.

In some embodiments, a collection of microbiota samples comprises at least one microbiota sample. In some embodiments a microbiota sample comprises 1, 2, 3, 5, 10, 15, 20, 25, 30, 35, 40, 45, 50, 100, or 1,000 or more samples.

In some embodiments, the first and second collections of microbiota samples are any two collections of microbiota samples that differ in a feature of radiation exposure but are otherwise comparable. In some embodiments, the first and second collections of microbiota samples are obtained from different host organisms. In some embodiments, the first and second collections of microbiota samples are obtained at from a same collection of hosts at different times. In some embodiments, the first and second collections of microbiota samples.

In some embodiments, a feature of radiation exposure comprises a dose of radiation exposure to a host from which a microbiota sample is obtained. In some embodiments, a dose of radiation exposure comprises between 0 and 1 Gy. In some embodiments, dose of radiation exposure comprises 1, 2, 3, 4, 5, 6, 7, 8, 9, or 10 Gy or more.

In some embodiments, a feature of radiation exposure comprises a duration of radiation exposure to a host from which a microbiota sample is obtained. In some embodiments, the duration is between 0 and 1 seconds. In some embodiments, the duration is 1, 2, 3, 4, 5, 6, 7, 8, 9, 10 or more seconds. In some embodiments, the duration is 1, 2, 3, 4, 5, 6, 7, 8, 9, 10 or more minutes. In some embodiments, the duration is 1, 2, 3, 4, 5, 6, 7, 8, 9, 10 or more hours. In some embodiments, the duration is 1, 2, 3, 4, 5, 6, 7, 8, 9, 10 or more days.

In some embodiments, a feature of radiation exposure comprises a duration of time post-exposure to a host from which a microbiota sample is obtained. In some embodiments, the duration is 1, 2, 3, 4, 5, 6, 7, 8, 9, 10 or more minutes. In some embodiments, the duration is 1, 2, 3, 4, 5, 6, 7, 8, 9, 10 or more hours. In some embodiments, the duration is 1, 2, 3, 4, 5, 6, 7, 8, 9, 10 or more days. In some embodiments, the duration is 1, 2, 3, 4, 5, 6, 7, 8, 9, 10 or more weeks.

In some embodiments, a feature of radiation exposure comprises a frequency of exposure to radiation to a host from which a microbiota sample is obtained. In some embodiments, a frequency of exposure to radiation comprises exposure of a host to radiation one or more times per second. In some embodiments, a frequency of exposure to radiation comprises exposure of a host to radiation one or more times per minute. In some embodiments, a frequency of exposure to radiation comprises exposure of a host to radiation one or more times per hour. In some embodiments, a frequency of exposure to radiation comprises exposure of a host to radiation one or more times per day. In some embodiments, a frequency of exposure to radiation comprises exposure of a host to radiation one or more times per week. In some embodiments, a frequency of exposure to radiation comprises exposure of a host to radiation one or more times per month. In some embodiments, a frequency of exposure to radiation comprises exposure of a host to radiation one or more times per year. In some embodiments, a frequency of exposure to radiation comprises exposure of a host to radiation one or more times per lifetime of a host.

In some embodiments, a feature of radiation exposure comprises a type of radiation exposure. Types of radiation exposure in accordance with the present invention include but are not limited to ionizing radiation, alpha radiation, beta radiation, cosmic radiation, neutron radiation, X-ray radiation, and gamma radiation or combinations thereof.

In some embodiments, identifying a microbial signature comprises any means that allows a signature correlated with a feature of radiation exposure to be identified. In some embodiments, identifying a microbial signature comprises identifying one or more levels in a first set of levels in the first collection of microbiota samples that are increased and/or decreased when compared to the second set of levels of the second collection of microbiota samples. In some embodiments, identifying a microbial signature comprises identifying levels of one or more DNA sequences that are increased and/or decreased in the first collection of microbiota samples when compared to the second collection of microbiota samples. In some embodiments, DNA sequences are identified by comparing semi-quantitative or quantitative real time PCR data for the first and second collections of microbiota samples. In some embodiments, DNA sequences are identified by performing cluster analysis on phylochip data generated from the first and second collections of microbiota samples. In some embodiments, identifying a microbial signature comprises identifying levels of one or more RNA transcripts that are increased and/or decreased in the first collection of microbiota samples when compared to the second collection of microbiota samples. In some embodiments, RNA transcripts are identified by comparing semi-quantitative or quantitative real time reverse transcriptase PCR data for the first and second collections of microbiota samples. In some embodiments, RNA sequences are identified by performing cluster analysis on microarray data generated from the first and second collections of microbiota samples. In some embodiments, identifying a microbial signature comprises identifying levels of one or more proteins that are increased and/or decreased in the first collection of microbiota samples when compared to the second collection of microbiota samples.

### Uses

The present invention encompasses the recognition that changes in microbial signature can be relied upon as a diagnostic tool to identify and characterize radiation exposure. As described herein, current tests for detecting radiation exposure either require extensive repeated testing or take upwards of three days post-exposure. There exists a need for more time sensitive tests with fewer required resources in nuclear event situations, allowing exposed individuals to be treated as soon as possible and non-exposed individuals to be released from a medical environment.

In some embodiments, the current invention provides methods of identifying and/or characterizing exposure to radiation comprising determining a microbial signature in a microbiota sample from an individual whose exposure to radiation is to be identified or characterized, and comparing it to a reference microbial signature that correlates with one or more features of exposure to radiation.

In some embodiments, an individual comprises any individual exposed to, suspected of being exposed to, and/or at risk of exposure to radiation.

In some embodiments, a reference microbial signature comprises any value that is correlated with a known feature of exposure to radiation. In some embodiments, a reference microbial signature comprises a microbial signature obtained from an individual who has not been exposed to radiation. In some embodiments, a reference microbial signature comprises a microbial signature from an individual who has been exposed to a known feature of radiation. In some embodiments, a reference microbial signature comprises a microbial signature from an individual who is comparable to the individual whose exposure to radiation is to be identified or characterized. In some embodiments, a reference microbial signature comprises a microbial signature that was obtained at a different time from the individual whose exposure to radiation is to be identified or characterized. In some embodiments, the different time occurred before exposure to radiation.

In some embodiments, a reference microbial signature is from a microbiota sample of an individual whose exposure to radiation is to be identified. In some embodiments, a reference microbial signature comprises a level and/or activity one or more microbes. In some embodiments, a reference microbial signature comprises a level and/or activity one or more microbes, wherein the level and/or activity of the one or more microbes remains substantially unchanged in response to radiation exposure.

In some embodiments, comparing a microbial signature in a microbiota sample from an individual whose exposure to radiation is to be identified or characterized, to a reference microbial signature comprises comparing microbial signatures obtained from two separate individuals. In some embodiments, comparing microbial signatures comprises comparing microbial signatures obtained from the same individual at separate time points. In some embodiments, comparing microbial signatures comprises comparing microbial signatures of the same microbial sample. In some embodiments, comparing microbial signatures comprises comparing relative levels and/or activities of two or more microbes. In some embodiments, comparing microbial signatures comprises comparing relative levels and/or activities of two or more microbes, wherein at least one first microbe (i.e., level and/or activity of at least one first microbe) remains substantially constant. In some such embodiments, comparing microbial signatures comprises comparing relative levels and/or activities of two or more microbes, wherein at least one second microbe changes.

### EXEMPLIFICATION

### Example 1

### Obtaining Microbial Samples of Irradiated Rats

In the following example, methods for obtaining microbial samples from irradiated rats are described. Male WAG/RijCmcr (Wistar) rats at 5 weeks of age are used. Littermate rats (n=5/group) are assigned at random to receive single or multiple fraction total body X-radiation of 10.0 Gy and 18.0 Gy, respectively. Total Body Irradiation is done with a posterior-anterior field at a dose rate of 1.95 Gy/min (Baker et al., Total body irradiation increases risk of coronary sclerosis, degeneration of heart structure and function in a rat model. Int J Radiat Biol, 85(12):1089-1100, 2009). Following irradiation rats in each group were housed with a maximum of three per cage for subsequent monitoring. Fresh fecal pellets are obtained from each rat prior to (day 0) and at days 4, 11 and 21 post irradiation. Pellets are homogenized in 1 ml PBS and 200 μl of homogenate was used for microbial DNA isolation using a QJAamp DNA Stool Mini Kit (Qiagen, Valencia, Calif.).

### Example 2

### Phylochip Analysis of Rat Microbiomes

In the following example, methods are described for quantifying microbial DNA using phylochip 16S rRNA gene microarrays. Microbial diversity and comparative community structure of rat fecal DNA samples is characterized by Second Genome Inc. (San Francisco, Calif.) using high-density G3 PhyloChip™ 16S rRNA microarray-based assays (PN49-0002A) and bioinformatic methods. Microbiota analysis is focused on calculating inter-sample distances and assessing significance of microbiome dissimilarity (Hazen et al. Deep-sea oil plume enriches indigenous oil-degrading bacteria. Science, 330(6001):204-8, 2010). Data analysis incorporates several separate stages; pre-processing and data reduction, summarization, normalization where needed, sample-to-sample distance metrics, ordination/clustering, sample classification, and significance testing.

Pre-processing and data reduction: To calculate a summary intensity for each feature on each array, 9 central pixels of individual features are ranked by intensity and 75% percentile is used. Probe intensities are background-subtracted and scaled to PhyloChip™ Control Mix™ (Standard-Scaling) (Second Genome, Inc., San Francisco). A hybridization score (HybScore) for an operational taxonomic unit (OTU) is calculated as a mean intensity of perfectly matching probes exclusive of maximum and minimum values. Data was reduced to consider taxa deemed present as described in Hazen et al. and filtered to taxa present in at least one sample or to taxa present in a majority of profiles of exactly one category and in zero other categories.

Sample-to-Sample Distance Function: All profiles are inter-compared in a pair-wise fashion to determine a dissimilarity score and to store it in a distance matrix. Distance functions are chosen to allow similar biological samples to produce only small dissimilarity scores. The Bray-Curtis Index utilizes taxon abundance differences across samples but employs a pair-wise normalization by dividing the sum of differences by the sum of all abundances.

Ordination, Clustering, and Classification Methods: Two-dimensional ordinations and hierarchical clustering map of samples as dendrograms are created to graphically summarize inter-sample relationships. To create dendrograms, distance matrix sample are clustered hierarchically using farthest neighbor distance, i.e. complete linkage. Non-Metric Multidimensional Scaling (NMDS) is a method of two-dimensional ordination plotting that is used to visualize complex relationships between samples. NMDS uses only rank order of dissimilarity values to position points relative to each other. Lists of significant taxa whose abundance characterizes each class is performed using Prediction Analysis for Microarrays which utilizes a nearest shrunken centroid method described in Tibshirani et al. in Pre-validation and inference in microarrays. (Stat Appl Genet Mol Biol, 1: Article 1, 2002).

Hybridization Scoring and Saturation: To calculate summary intensities for each feature on each array, 9 central pixels of individual features are ranked by intensity and 75% percentile is used as probe intensity. Probe intensities are background-subtracted and scaled via Standard Scaling to PhyloChip™ Control Mix (Second Genome Inc., San Francisco) so that mean probe intensity of all probes complimentary to any target in the control mix will equal 10,000 units. Since the same concentration of spike mix is added to each PhyloChip assay, scaled probe intensities are directly comparable to each other across arrays. When a probe's scaled intensity changes from array-to-array it indicates a change in target DNA concentration. The summary score for an operational taxonomic unit (OTU) is calculated as a mean intensity of perfectly matching probes exclusive of the maximum and minimum. These trimmed means can theoretically range from 0 to 65,536, but in real microbiome samples we commonly observe a range from ˜100 to ˜17,000. A common practice with microarray data is to logarithmically transform scores so that variance is constant over a broad concentration range. Log base 2 of scores was used which, for example, converts 100 to 6.644 and 17,000 to 14.053. In some applications, floating point numbers are difficult to work with so as a final step we multiply by 1000 to achieve integer HybScores such as 6,644 or 14,053. In supplementary material for Hazen et al., DNA from 26 different taxa were applied to G3 PhyloChip assays across a range of concentrations from 0 to 480 pM. HybScores correlated well with concentration (r=0.941) across the entire range but a slight slope reduction was observed for 8 of 26 taxa above HybScores of 15,000. In our experimental data, no HybScores were observed over the 15,000 threshold.

### Example 3

### Quantitative PCR of Microbial 16S rRNA Genes

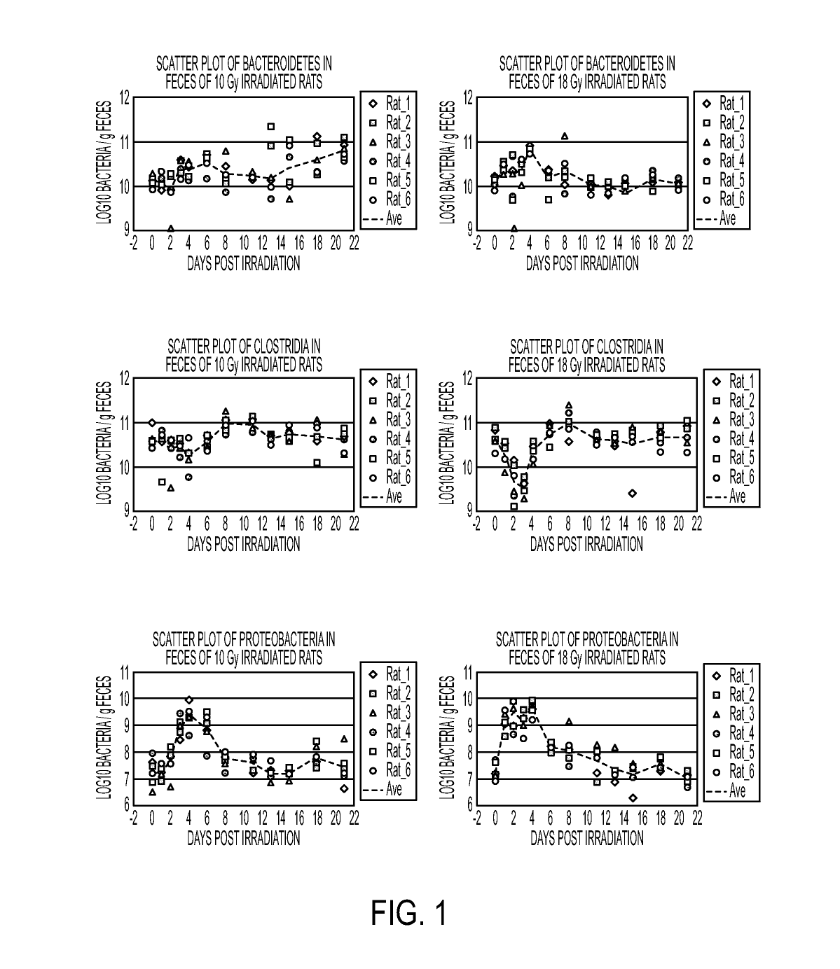

In the following example, methods are described for quantifying microbial levels via quantitative PCR of 16S rRNA gene sequences. Isolated DNA samples are subjected to quantitative PCR using an iCycler (Bio-Rad, Hercules, Calif.) or any other quantitative PCR machine for microbial population enumeration. PCR reaction mixture consists of 50% iQ SYBR Green Supermix (Bio-Rad), 0.4 μM forward and reverse primers, and 3.8% template solution in RNase/DNase free water. Primer combinations shown allow for detection of bacterial taxons indicated (table 1) or biomarker (table 2) indicated. A paired Students t-test was used to find significant differences among variables in qPCR data. PCR data variance is shown in representative scatter plots (FIG. 1).

TABLE 1
16S Primer Pairs for Bacterial Taxa

| Taxon | | | |
|---|---|---|---|
| Identified | Gene | Orientation | Sequence |
| Bacteroidetes | BactF285 | Forward | GGTTCTGAGAGGAGGTCCC |
| | UniR338 | Reverse | GCTGCCTCCCGTAGGAGT |
| Clostridia | UniF338 | Forward | ACTCCTACGGGAGGCAGC |
| | CcocR491 | Reverse | GCTTCTTAGTCAGGTACCGTCAT |
| Proteobacteria | Uni515F | Forward | GTGCCAGCMGCCGCGGTAA |
| | Ent826R | Reverse | GCCTCAAGGGCACAACCTCCAAG |

TABLE 2
Primer Pairs for Biomarkers

| Biomarker | Orientation | Sequence |
|---|---|---|
| StableF | Forward | TTCGCTTCTCTTCGTATGCGGC |
| StableR | Reverse | TCTTCACACACGCGGCATGGC |
| DownF | Forward | CGCGTGGGTAACCTGCCCTG |
| DownR | Reverse | CGCGGGTCCATCCTATACCGCA |
| AcuteF | Forward | TCGGGCCTCTTGCCATCGGA |
| AcuteR | Reverse | CCGGTTAACGCTTGCACCCCT |
| ChronicF | Forward | CTGGGATGGACCTGCGGTGT |
| ChronicR | Reverse | TTACGAGCCGAAACCCTTCTTCAC |

### Example 4

### Identifying Candidate Biomarkers of Prior Radiation Exposure

In the following example, candidate biomarkers for prior exposure to radiation are identified. 16S ribosomal RNA (rRNA) gene sequences are thought to be unique to each eubacteria taxon and changes in quantity of 16S rRNA genes across total DNA extraction products are thought to be indicative of changes in species abundance.

Microbes from feces were obtained using techniques described in Example 1 from five independent rats at all time points (0, 4, 11 and 21 days) after exposure to 10 Gy single-fraction total body irradiation for analysis. Microbial diversity and comparative community structure of rat fecal DNA samples were characterized using G3 PhyloChip 16S microarray-based assay and bioinformatic methods described in Example 2.

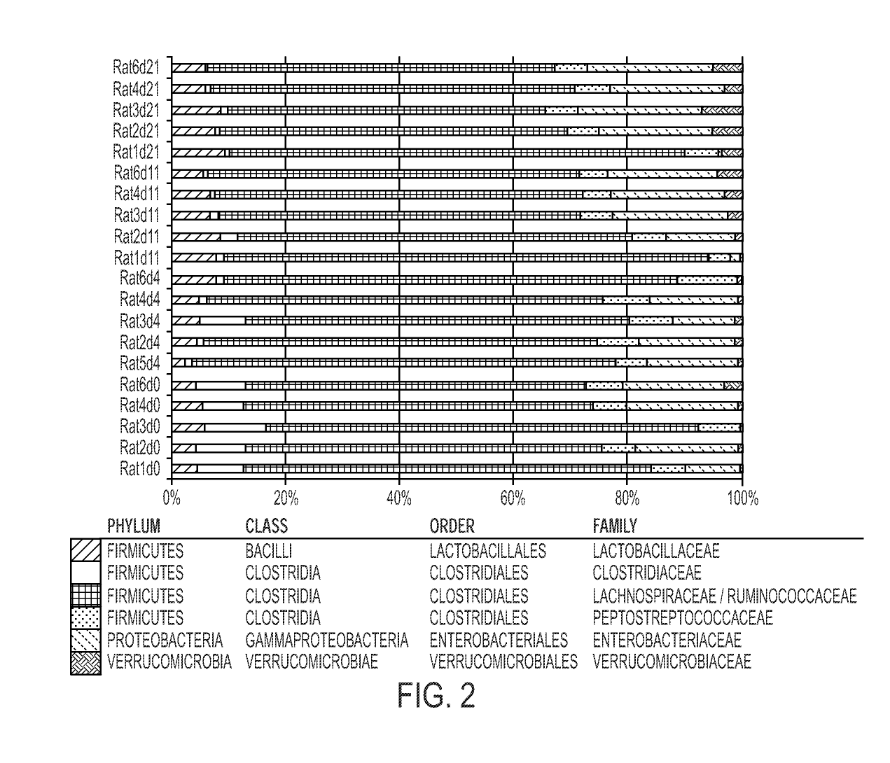

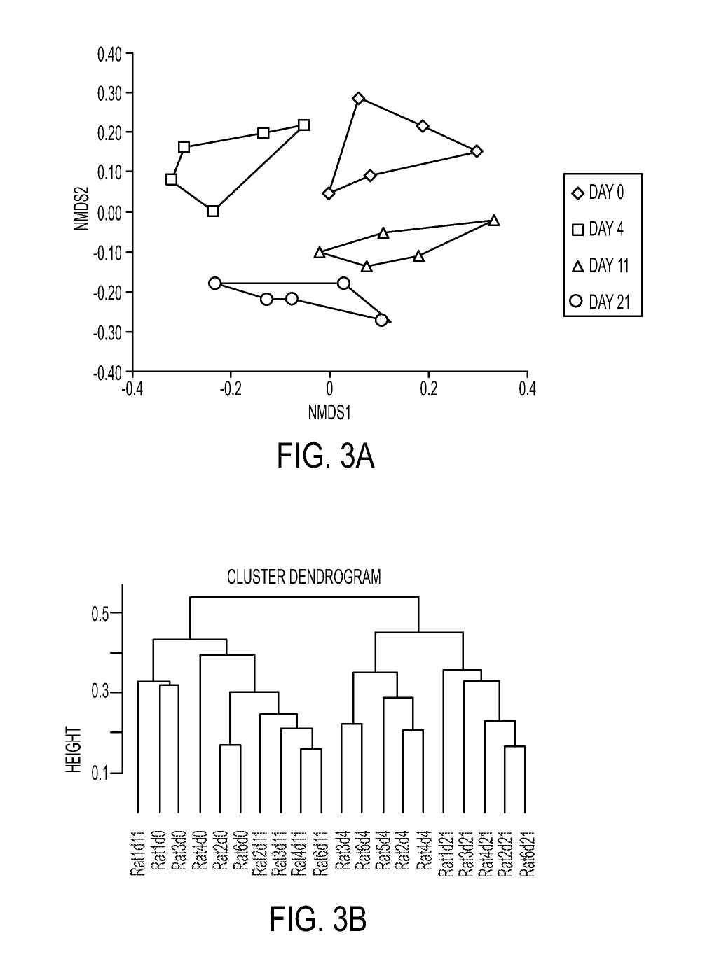

Members of Firmicutes and Proteobacteria phyla were found to be the most abundant microbiota present in the feces (FIG. 2). Microbiota analysis focused on calculating inter-sample distances and assessing significance of microbiome dissimilarity without use of pre-exposure controls. This aspect of the analysis is very important for translation ultimately to a radiation triage situation in which pre-exposure controls for each individual will not be available. A total of 7,484 bacterial operational taxonomic units (OTUs) were detected in at least one sample. The Adonis test demonstrated bacterial communities were more dissimilar across days than they were within the same day (p<0.001). Samples separated more distinctly by day than by rat when all taxa present in at least one sample was considered using the Bray-Curtis dissimilarity measurement (FIG. 3). Hierarchical clustering as dendrograms using the complete linkage method revealed a close relationship at days 0, 4, 11 and 21 (FIG. 3).

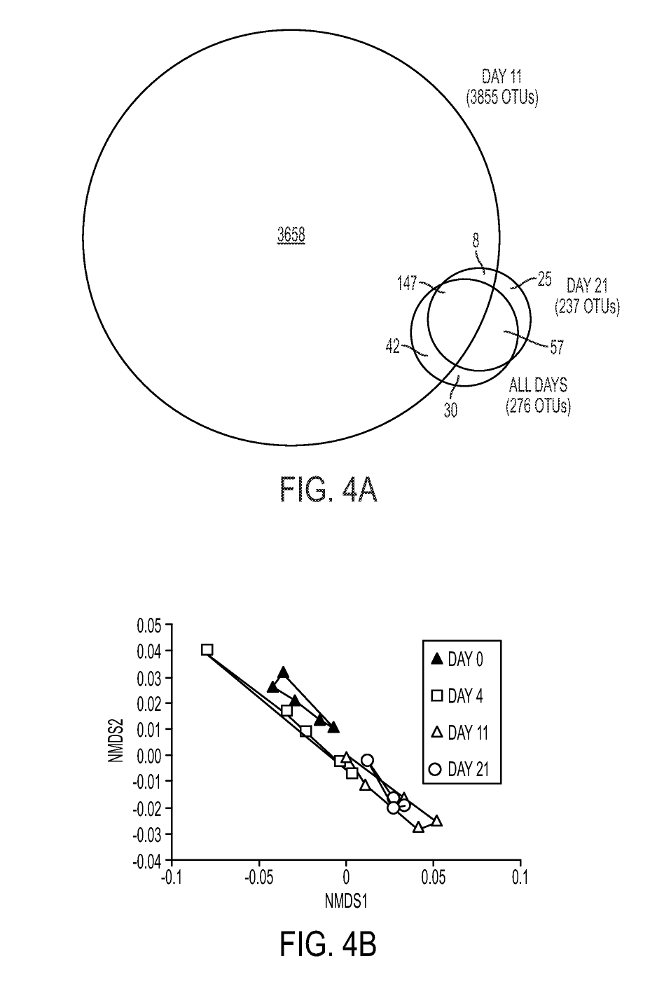

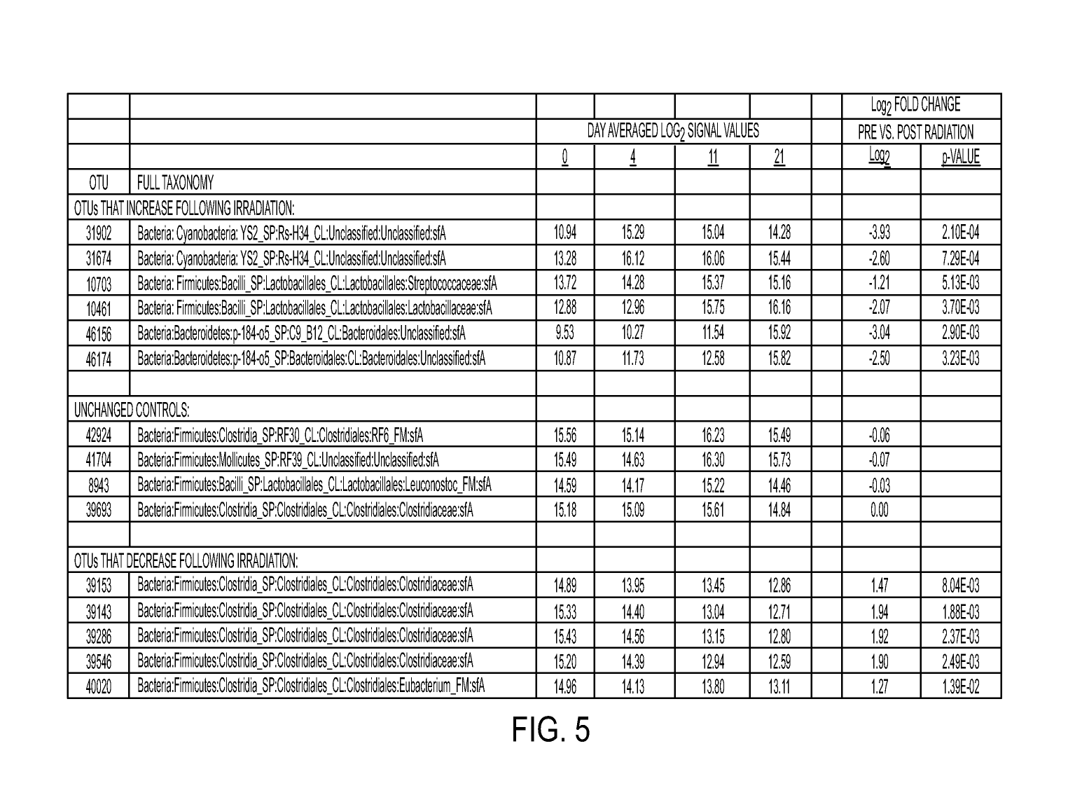

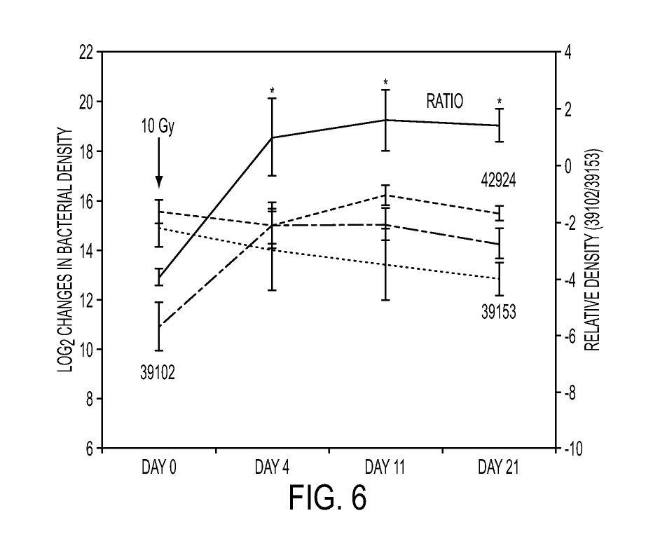

In order to find candidate biomarkers of prior radiation exposure, OTUs were identified that exhibited changes in abundance that were persistent from day 4 through day 21 post irradiation. Abundance levels of 276 OTUs were found that were changed at days 4 through 21 when the number of false discoveries were limited to 5 (total), as estimated by the q-value (All Days, FIG. 4) (Turnbaugh, P. J. et al. “The effect of diet on the human gut microbiome: a metagenomic analysis in humanized gnotobiotic mice.” Sci Transl Med, 1(6):6ra14, 2009). These 276 OTUs were then compared with 3855 and 237 OTUs that were significantly altered on days 11 and 21 as compared to background (Days 11 and 21, FIG. 4). A common set of 147 OTUs were found between these three comparisons and were used as an initial list of biomarker candidates. Ordination using these 147 OTUs separated these data points by days post irradiation using nonmetric multidimensional scaling (p<0.001). Representative OTUs from this list are shown in FIG. 5. Of 276 OTUs that showed significant changes between background, day 0, and days 4 through 21, OTUs were further selected that exhibited a persistent decrease in abundance following irradiation (FIG. 5). Even though changes in abundance of these OTUs were not as large as the top 147 candidates discussed above, they represented a distinctly different phenotype. 165 Bacteroidales, Lactobacillaceae and Streptococcaceae OTUs were found with increased expression following radiation exposure, 142 Clostridiaceae and Peptostreptococcaceae OTUs were found with unchanged abundance that may serve as internal controls, and 47 separate Clostridiaceae OTUs with decreased expression were identified. A complete listing of OTUs that increased, decreased or were unchanged following irradiation is provided in the Appendix. Results from microarray studies, described herein, are also specific to individual bacteria. For example, abundance of OTU 31902 (Cyanobacteria) increased, OTU 39153 (Clostridia) decreased, and OTU 42924 (Clostridia) was unchanged in the 4-21 day period post radiation exposure (FIG. 6). The “increased/decreased” ratio of 31902/39153 increased from −4 to +2 log₂ difference indicating a 64 fold change at days 4, 11, and 21 post irradiation (FIG. 6), and may also be used as a possible biomarker of prior radiation exposure. Use of a ratio in developing intestinal microbiota as biomarkers for radiation biodosimetry may be advantageous as preexposure samples will not be available during or after a radiological device being detonated.

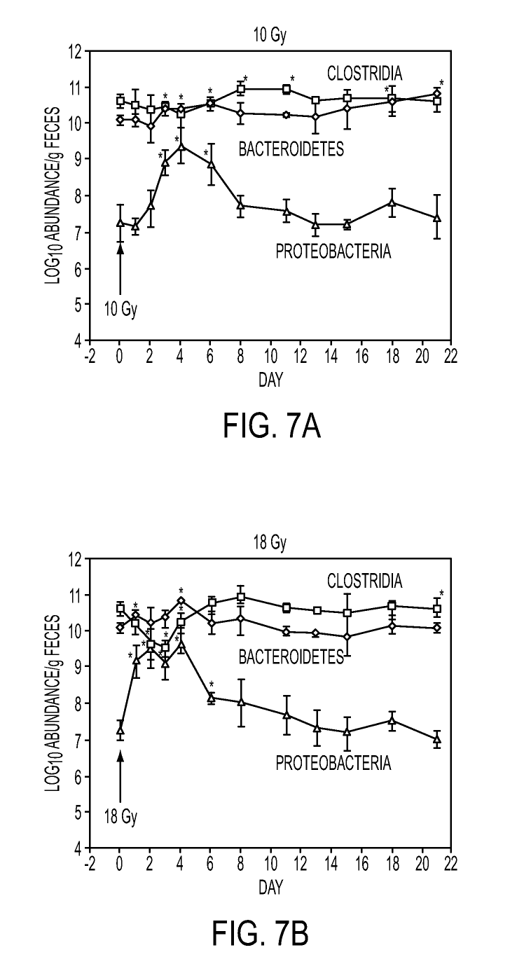

To determine whether specific groups of bacteria identified by microarray analysis are detectable by an independent method, feces were also analyzed for expression of 16S rRNA in selected groups of intestinal microbiota using qPCR. Abundance of Proteobacteria increased almost 1000 fold 4 days following 10 Gy total body irradiation and then returned to control values (FIG. 7A). Abundance of Clostridia and Bacteroidetes was less affected over this period. The results herein suggest particular microbial taxa, e.g. order or family, whose abundance are unaffected by radiation may serve as internal controls (FIG. 5, FIG. 7A). In these studies, primers for 16S rRNA detected over 100 separate members in each bacterial group.

This OTU level analysis of feces of irradiated rats showed changes in abundance levels of 212 genomically distinct bacteria, of which 59 (12 increased and 47 decreased following irradiation) are found in normal human feces. 16S rRNA levels in 98 intestinal microbiota unaffected by radiation serve as internal controls. Intestinal microbiota affected by irradiation provide a sustained level of reporting signals that persist at least 21 days following exposure to radiation. The ‘increased/decreased’ ratio of two individual bacteria increased 64 fold at day 21 compared with Day 0. The results of the present disclosure suggest these bacteria have utility as a biomarker of prior exposure to ionizing radiation, as demonstrated by PCR results described herein. These biomarkers may also be indicative of early gastrointestinal system injury following fractionated therapeutic radiation. Evidence that abundance of copies of the 16S rRNA gene in response to ionizing radiation is robust. The present disclosure therefore indicates that gene abundance signatures are likely to be translatable from discovery platforms to developing “fieldable” assay platforms more suitable for practical biodosimetry.

### Example 5

### Dose-Response Studies

In the following example, dose response studies to determine the effect of differing radiation doses are described. To determine impact of radiation dose, studies were conducted using six fractionated exposures totaling 18 Gy over a three-day period to model therapeutic radiation used clinically. The method of collecting microbial specimens from irradiated rats described in Example 1 was used. Quantitative PCR of microbial 16S rRNA genes using the method described in Example 3 was done to quantitate amount of bacteria from each taxon. Results described herein show 18 Gy irradiation induced a prolonged increase in Proteobacteria over 5 days (FIG. 7B), as compared to 3 days observed after 10 Gy (FIG. 7A). Further, 18 Gy irradiation induced a 10 fold reduction in Clostridia at days 1-3 that was not observed with 10 Gy irradiation. Increases in Proteobacteria at two days post 18 Gy irradiation correspond with equivalent responses observed at four days post 10 Gy irradiation since 6 fractions of the 18 Gy regimen were administered over three days instead of one.

### Example 6

### Design of Biomarker Assay of Prior Radiation Exposure

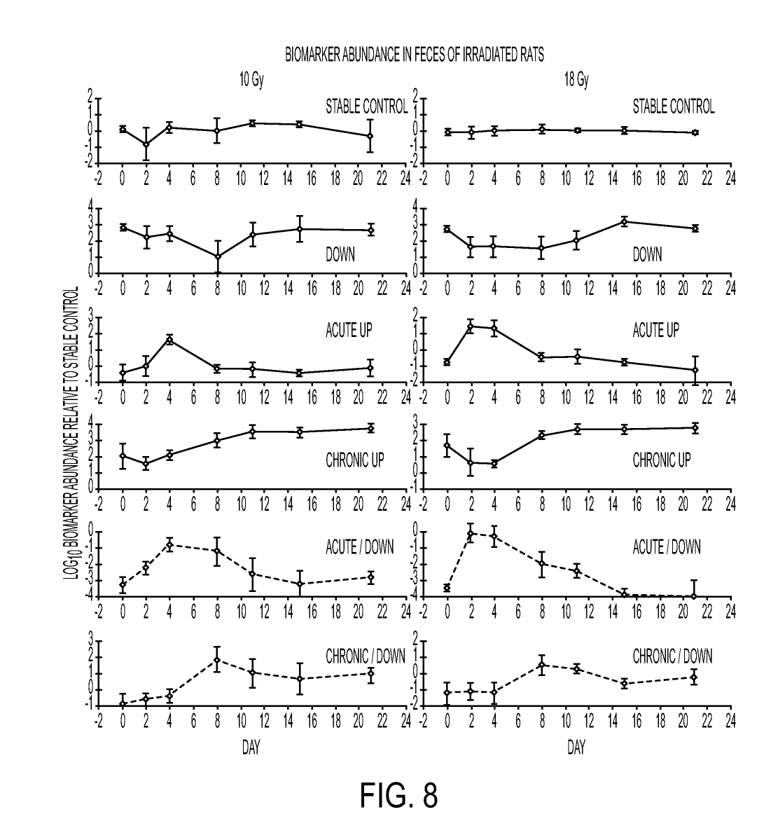

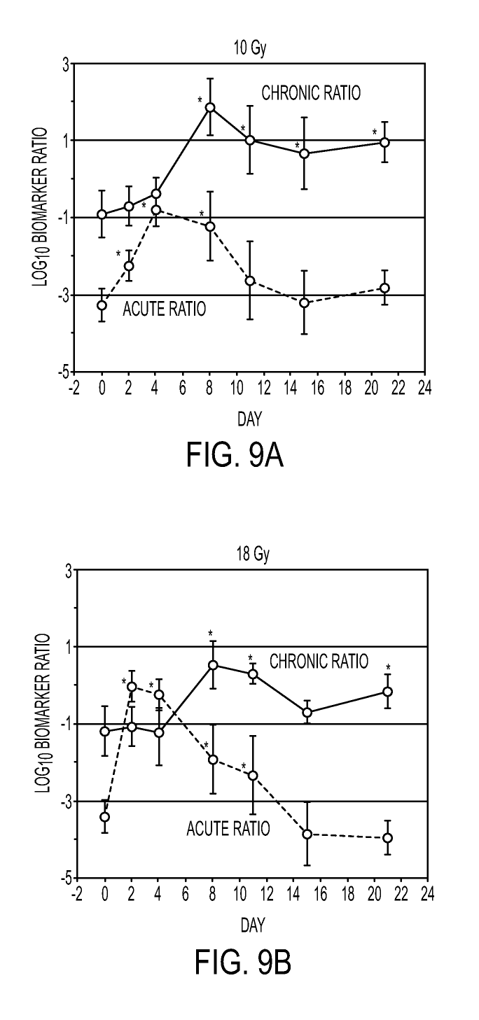

In the following example, development of an assay to detect microbial signatures is described. Guided by biomarker OTUs discovered using PhyloChip analysis described in Example 4, PCR primers were designed for quantitative PCR of microbial 16S rRNA genes using the method described in Example 3 to confirm irradiation induced changes in biomarker abundance in irradiated rats treated according to the protocol described in Example 1. Biomarkers were identified that were stable, decreased, acutely increased (within the first week), and chronically increased (for more than 21 days) following irradiation (FIG. 8). The biomarker ratio “acute increase/decrease” was increased from 2 to 8 days following 10 and 18 Gy irradiation, while the ratio “chronic increase/decrease” was increased from 8 to 21 days post irradiation (FIGS. 9A and 9B). The present disclosure therefore confirms existence of individuals or groups of microbes that can serve as biomarkers of prior radiation exposure.

### Example 7

### Impact of Factors Other than Radiation on Microbial Populations

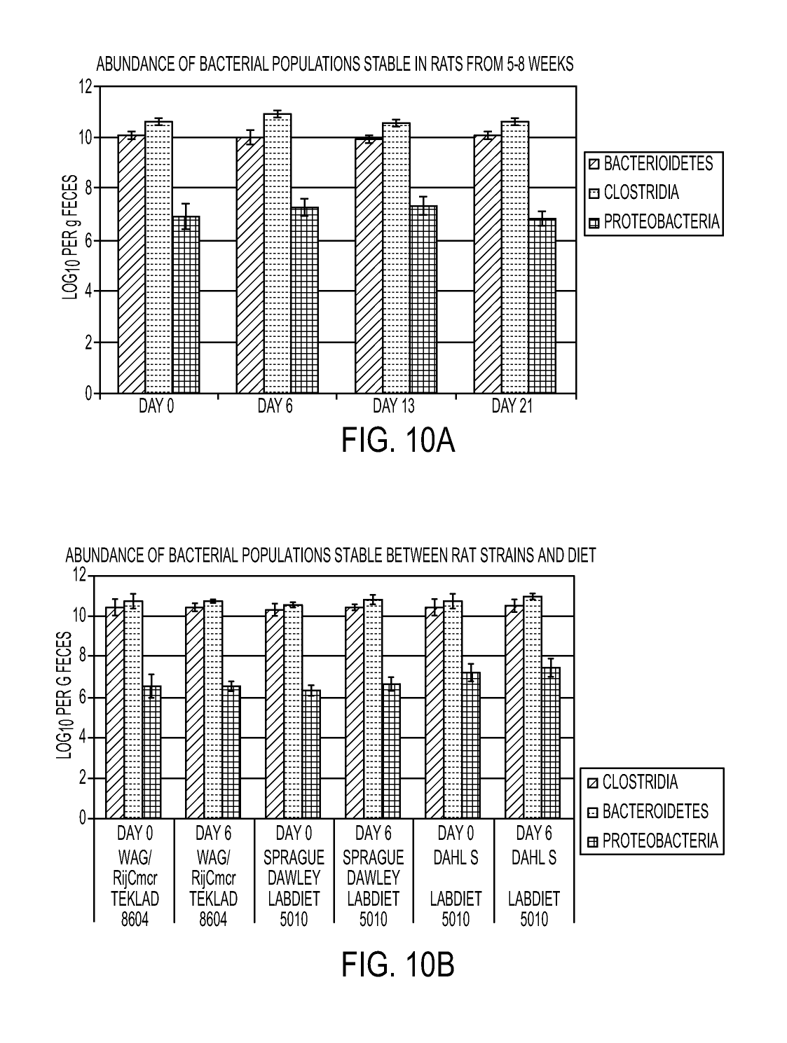

In the following example, impact of factors other than radiation on microbial populations is assessed to determine impact of age on abundance of intestinal microbial populations feces were collected from non-irradiated 5 week old rats over 21 days according to the protocol described in Example 1. Abundance of Bacteroidetes, Proteobacteria and Clostridia was assayed using the method for quantitative PCR of microbial 16S rRNA genes described in Example 3. Levels of Bacteroidetes, Proteobacteria and Clostridia did not change over 21 days of study. Thus abundance of three major (>90% of microbiota) groups of bacteria affected by radiation were unchanged over time in rats not exposed to radiation (FIG. 10A). The present disclosure therefore indicates that genetic background and age do not appear to exert changes in abundance for multiple bacterial taxa including Bacteroidetes, Proteobacteria and Clostridia in control rats not exposed to radiation. Abundance of multiple intestinal bacterial taxa were also unaffected by diet in control rats.

To determine impact of genetic background and diet on abundance of microbial populations present in rat, feces were collected from inbred WAG/RijCmcr rats fed Teklad 8604 chow, outbred Sprague Dawley rats fed LabDiet 5010 chow and inbred Dahl S rats fed LabDiet 5010 chow. All three rat strains studied are permanently maintained on these diets. Abundance of Bacteroidetes, Proteobacteria and Clostridia were assayed using the method for quantitative PCR of microbial 16S rRNA genes described in Example 3. Levels of Bacteroidetes, Proteobacteria and Clostridia in these three rat strains was comparable and stable over a 6 day study period. The present disclosure therefore indicates that strain and diet do not exert an effect on these three bacterial populations present in rat feces (FIG. 10B).

### Example 8

### Human and Rat Similarities

In the following example, human gastrointestinal tract microbiomes are studied to identify similarities with data found in rats. 6 human fecal samples were analyzed using G3 PhyloChips. The present disclosure reveals that, when comparing this data to data for rats from Example 4, all 47 OTUs found to decreased in rats are present in humans, 98 of 142 stable OTUs in rat are present in humans, and 12 of 165 OTUs that increased in rat are found in humans. The present disclosure therefore indicates that these 157 OTUs form a microbial signature that correlates with and appears to be diagnostic of prior exposure to radiation.

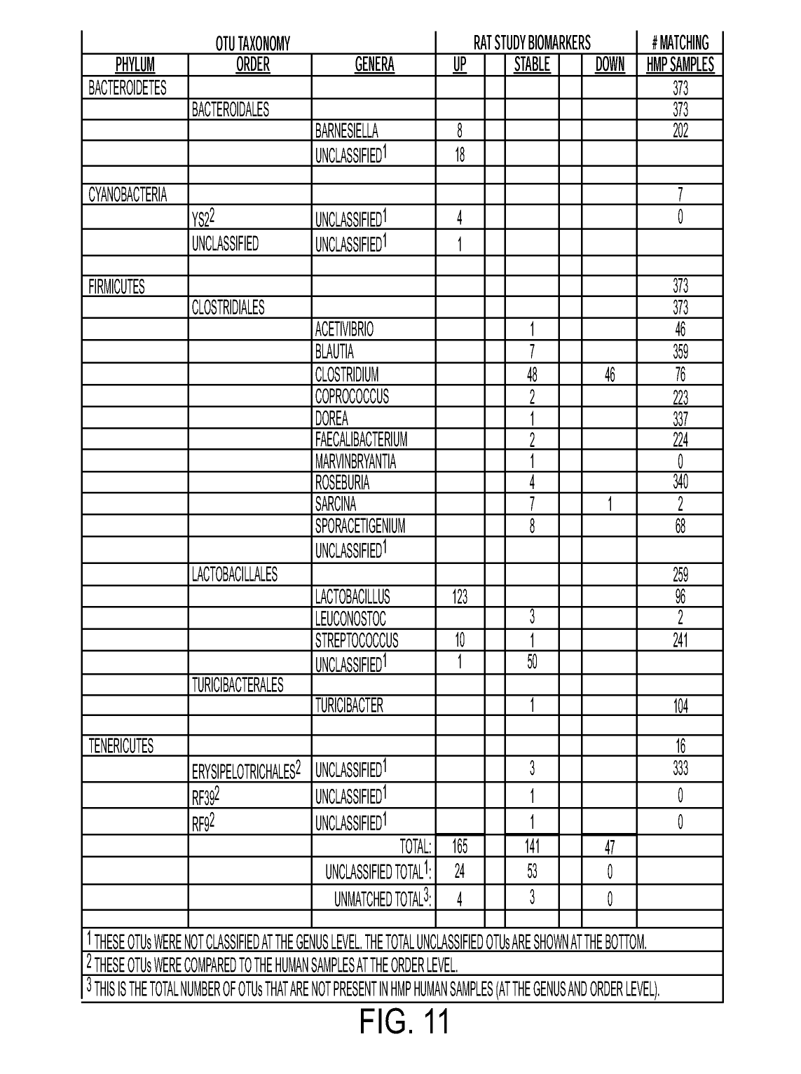

Rat-to-human analysis was further broadened by comparing rat OTUs from Example 4 to bacterial taxa detected in 373 stool samples collected during the human microbiome project (http://www.hmpdacc.org/data_browser.php). Rat fecal OTUs were binned at genus-level to match pyrosequencing data from human samples (FIG. 9). 47 OTUs that decreased in rats were mapped to two genera: Clostridium and Sarcina (both are present in humans). Eighty nine of 142 stable OTUs were mapped to 14 genera in the Firmicutes phylum (FIG. 11) of which 13 are present in humans. One hundred and forty one of 165 OTUs that increased in rat were mapped to three genera: Barnesiella, Lactobacillus, and Streptococcus, and all three are present in humans. The present disclosure therefore indicates that more than 96% of classified rat biomarkers are matched to bacterial genera present in humans.

### Example 9

### Determining Minimum Dose Detectable by Microbial Signatures

The following example describes an experiment to determine a minimum dose of radiation detectable using microbial signatures. The following experimental method allows determination of a minimum dose detectable by biomarkers of the present invention. Male rats (n=50) and then mixed breed or beagle dogs (n=10) are irradiated with a single exposure to 350 kVp X-rays (n=5/group) over a range 1-10 Gy to determine an earliest response time and duration of effect (1-15 days). A dose of 1 Gy is first used and dosage is then progressively increase in 1 Gy increments. A lowest dose and earliest response from rat and dog is confirmed. Feces is collected daily and analyzed with a microarray analysis signature specific for radiation exposure described herein. Findings are confirmed using qPCR. This method will allow identification of a dose- and time-response characteristics for intestinal microbiota to detect prior exposure to ionizing radiation.

It is expected that changes in intestinal microbiota following irradiation will be detectable as early as 24 hours after exposure with radiation signatures persisting for at least 15 days. It is expected that prior exposure at radiation levels as low as 2 Gy, a dose considered important for triage purposes, will be detectable. Detection of exposure to levels greater than 2 Gy will be important to determine medical treatment. Onset of diarrhea in dogs is expected 4-8 hours after radiation exposures above 4 Gy. Animals will receive medical support as needed. Monitoring gastrointestinal symptoms in dogs allows determine of whether they are correlated with microbiota profiles. Response of oral cavity microbiota to ionizing radiation is an alternate approach.

### Example 10

### Determine Impact of Health Status on Intestinal Microbiota after Irradiation

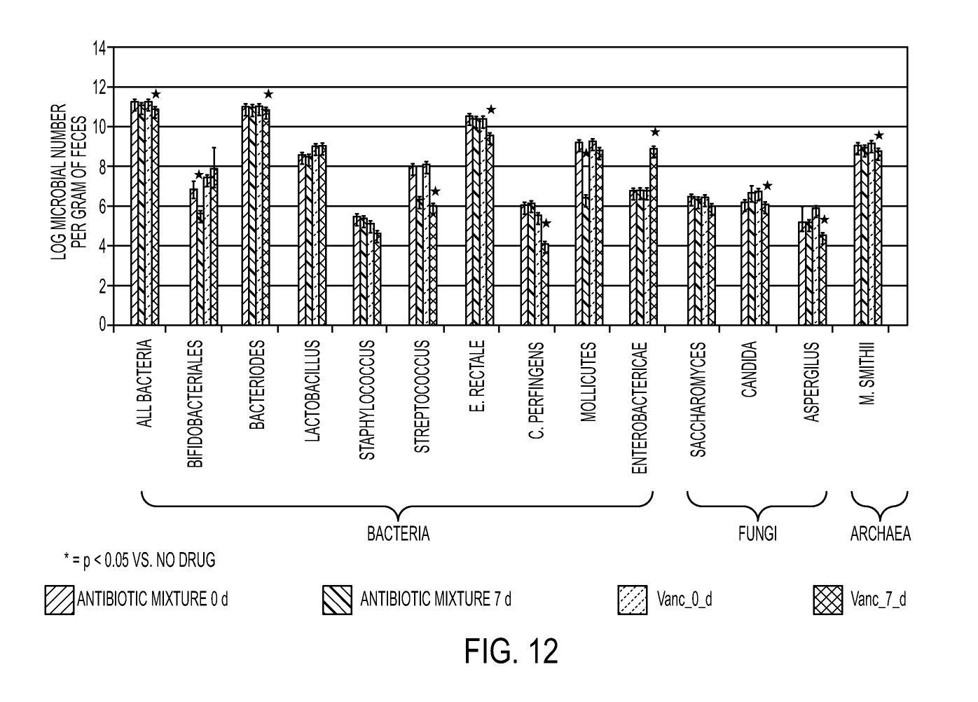

The following example describes experiments to determine the effect of health status on microbial signatures. Antibiotic use throughout the US population is common (Grijalva et al. Antibiotic prescription rates for acute respiratory tract infections in US ambulatory settings. JAMA, 302:758-66, 2009). Azithromycin is now the most commonly prescribed macrolide for acute respiratory tract infections and otitis media. Immediately following nuclear detonation, supportive care including antibiotics can improve the prognosis for some irradiated casualties (DiCarlo et al. Radiation injury after a nuclear detonation: medical consequences and the need for scarce resources allocation. Disaster Med Public Health Preparedness, 5:S32-S44, 2011). Orally administered vancomycin, an antibiotic in current clinical use, and a mixture of streptomycin, bacitracin polymyxin B and neomycin (Croswell et al. Prolonged impact of antibiotics on intestinal microbial ecology and susceptibility to enteric Salmonella infection. Infect Immun., 77:2741-53, 2009) alter abundance of intestinal microbiota present in feces of inbred Wistar rats (FIG. 12). An essentially equivalent response was seen in outbred Sprague Dawley and inbred Dahl S rats.

The following experimental method allows determination of whether microbes affected by antibiotics are the same as those affected by radiation by examining impact of pre-existing use of azithromycin on abundance of intestinal microbiota following exposure to radiation. Three groups are irradiated with a single fraction exposure with X-rays and their feces are examined for changes in intestinal microbiota signature. Rats (n=15) and dogs (n=5) in a first group are treated with azithromycin (15-60 mg/kg/day) for 7 days prior to irradiation. A second group (rats, n=15 and dogs, n=5) are treated 2-15 days post irradiation with ciprofloxacin, (15-30 mg/kg/day), and a third group (rats, n=15) receives a skin wound post irradiation. Findings are confirmed using qPCR.

For individuals receiving a skin wound, twenty-four hours prior to study, hair from rat dorsal surfaces is removed under light anesthesia (isoflurane inhalation) using electric clippers. On the day of the experiments (day 0), rats are placed two at a time in a custom-made acrylic jig for irradiation or sham treatment. Within one hour after irradiation, rats are anesthetized (isoflurane inhalation) and skin decontaminated according to standard surgical procedure. Under sterile conditions, two full-thickness circular wounds are made on rats on their backs above the spinal cord within the center of the radiation field using an 8 mm diameter punch biopsy. Hemostasis is achieved by blotting wounds with sterile gauze. An analgesic, Rimadyle (carprofen, Pfizer, USA), is injected at 5 mg/kg subcutaneously every 12 h for two days. Wounds are left uncovered for the duration of the study. The skin wound model has been described in Jourdan et al. (“Deposition is Diminished in Irradiated Skin in an Animal Model of Combined Radiation and Wound Skin Injury.” Radiat Res. 176:636-48, 2011).

It is expected that both antibiotics affect specific microbiota present in feces. This method will enable determination of an extent to which antibiotics distort radiation signatures. It is expected that skin wound injury may also exert an effect on intestinal microbiota following radiation exposure. Granulocyte macrophage colony-stimulating factor to stimulate hematopoiesis may be included as an alternate approach to antibiotics. The described method will allow determination of whether these underlying health conditions interfere with the response of intestinal microbiota to radiation.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

### APPENDIX

TABLE 1
OTUs increased following irradiation
Log2 Fold
Change

| | Day Averaged Log2 | | | | | |
|---|---|---|---|---|---|---|
| | Signal Values | | | Pre vs. Post Radiation | | |
| OTU | 0 | 4 | 11 | 21 | Log2 | p-value |
| 31902 | 10.94 | 15.29 | 15.04 | 14.28 | −3.93 | 2.10E−04 |
| 46156 | 9.53 | 10.27 | 11.54 | 15.92 | −3.04 | 2.90E−03 |
| 31674 | 13.28 | 16.12 | 16.06 | 15.44 | −2.60 | 7.29E−04 |
| 46174 | 10.87 | 11.73 | 12.58 | 15.82 | −2.50 | 3.23E−03 |
| 45878 | 11.49 | 11.91 | 13.21 | 16.08 | −2.25 | 4.11E−03 |
| 46045 | 11.88 | 12.80 | 13.48 | 16.11 | −2.25 | 4.54E−03 |
| 46464 | 12.29 | 12.89 | 14.10 | 16.40 | −2.17 | 1.30E−03 |
| 46118 | 12.18 | 12.71 | 13.79 | 16.44 | −2.13 | 3.96E−03 |
| 46123 | 12.13 | 12.62 | 13.76 | 16.40 | −2.13 | 2.49E−03 |
| 10509 | 13.65 | 13.87 | 16.56 | 16.89 | −2.12 | 2.91E−04 |
| 10461 | 12.88 | 12.96 | 15.75 | 16.16 | −2.07 | 3.70E−03 |
| 46422 | 12.65 | 13.11 | 14.43 | 16.27 | −1.95 | 3.93E−03 |
| 10021 | 13.60 | 14.10 | 16.34 | 16.17 | −1.94 | 2.64E−03 |
| 46592 | 12.49 | 12.64 | 14.17 | 16.47 | −1.93 | 5.86E−03 |
| 45539 | 11.71 | 12.30 | 13.13 | 15.45 | −1.92 | 5.88E−03 |
| 46370 | 12.58 | 13.13 | 14.21 | 16.15 | −1.92 | 1.39E−03 |
| 9685 | 14.04 | 14.20 | 16.69 | 16.92 | −1.90 | 5.63E−04 |
| 46656 | 13.10 | 13.68 | 14.67 | 16.57 | −1.87 | 2.77E−03 |
| 10405 | 13.39 | 13.33 | 16.06 | 16.37 | −1.87 | 1.50E−03 |
| 10597 | 13.63 | 13.84 | 16.15 | 16.39 | −1.83 | 4.48E−04 |
| 10577 | 14.05 | 14.30 | 16.60 | 16.70 | −1.82 | 6.08E−04 |
| 10309 | 14.27 | 14.59 | 16.81 | 16.85 | −1.81 | 4.71E−04 |
| 10155 | 14.32 | 14.67 | 16.80 | 16.87 | −1.79 | 2.54E−04 |
| 10849 | 14.51 | 14.92 | 17.02 | 16.89 | −1.77 | 6.51E−04 |
| 45635 | 13.29 | 13.86 | 14.78 | 16.54 | −1.77 | 4.21E−03 |
| 10632 | 14.43 | 14.98 | 16.86 | 16.74 | −1.76 | 2.94E−04 |
| 32070 | 13.31 | 15.33 | 15.28 | 14.57 | −1.75 | 5.64E−03 |
| 46404 | 13.15 | 13.97 | 14.50 | 16.22 | −1.75 | 2.23E−03 |
| 46326 | 13.27 | 13.71 | 14.86 | 16.47 | −1.75 | 1.72E−03 |
| 46044 | 12.74 | 13.21 | 14.33 | 15.87 | −1.73 | 2.21E−03 |
| 10168 | 13.94 | 13.71 | 16.47 | 16.77 | −1.72 | 2.38E−03 |
| 46556 | 13.29 | 13.65 | 14.74 | 16.62 | −1.72 | 3.99E−03 |
| 10516 | 14.15 | 14.31 | 16.62 | 16.66 | −1.71 | 5.78E−04 |
| 9808 | 14.44 | 14.83 | 16.82 | 16.81 | −1.71 | 2.61E−04 |
| 10830 | 14.02 | 13.88 | 16.50 | 16.67 | −1.66 | 2.00E−03 |
| 10768 | 14.03 | 15.36 | 15.97 | 15.72 | −1.66 | 1.87E−04 |
| 10001 | 14.31 | 14.60 | 16.74 | 16.49 | −1.64 | 3.93E−03 |
| 45933 | 12.98 | 13.10 | 14.39 | 16.31 | −1.62 | 5.65E−03 |
| 10232 | 13.17 | 14.04 | 15.20 | 15.14 | −1.62 | 1.12E−03 |
| 10250 | 13.17 | 14.04 | 15.20 | 15.14 | −1.62 | 1.12E−03 |
| 10596 | 13.17 | 14.04 | 15.20 | 15.14 | −1.62 | 1.12E−03 |
| 35067 | 14.82 | 15.60 | 17.20 | 16.53 | −1.62 | 4.28E−03 |
| 9680 | 14.52 | 14.56 | 16.86 | 16.98 | −1.61 | 3.48E−03 |
| 10858 | 14.93 | 15.45 | 17.20 | 16.96 | −1.61 | 2.55E−03 |
| 10122 | 14.50 | 14.61 | 16.86 | 16.81 | −1.59 | 9.20E−04 |
| 10078 | 14.90 | 15.30 | 17.13 | 17.02 | −1.58 | 2.77E−04 |
| 9710 | 14.57 | 14.79 | 16.85 | 16.82 | −1.58 | 6.19E−04 |
| 31568 | 14.91 | 16.62 | 16.73 | 16.05 | −1.56 | 2.06E−03 |
| 45735 | 12.79 | 13.32 | 14.26 | 15.46 | −1.55 | 3.68E−03 |
| 10520 | 14.51 | 14.84 | 16.77 | 16.55 | −1.54 | 2.36E−03 |
| 10148 | 14.70 | 14.64 | 16.99 | 17.07 | −1.54 | 1.58E−03 |
| 9839 | 13.55 | 14.49 | 15.45 | 15.31 | −1.53 | 1.76E−04 |
| 31504 | 14.27 | 15.84 | 16.14 | 15.40 | −1.52 | 5.27E−03 |
| 45848 | 13.75 | 14.45 | 15.08 | 16.27 | −1.52 | 3.88E−03 |
| 45505 | 13.75 | 14.45 | 15.08 | 16.27 | −1.52 | 3.88E−03 |
| 46054 | 13.75 | 14.45 | 15.08 | 16.27 | −1.52 | 3.88E−03 |
| 10701 | 14.58 | 14.46 | 16.86 | 16.97 | −1.52 | 2.20E−03 |
| 10474 | 14.56 | 14.45 | 16.81 | 16.91 | −1.50 | 3.00E−03 |
| 10135 | 14.78 | 14.78 | 17.02 | 17.05 | −1.50 | 1.32E−03 |
| 10356 | 14.76 | 15.13 | 16.93 | 16.71 | −1.49 | 1.43E−03 |
| 9747 | 14.77 | 15.03 | 16.94 | 16.79 | −1.49 | 7.73E−04 |
| 9883 | 14.43 | 14.76 | 16.62 | 16.35 | −1.48 | 5.48E−04 |
| 10017 | 14.54 | 14.67 | 16.71 | 16.66 | −1.48 | 1.79E−03 |
| 10140 | 14.61 | 14.63 | 16.83 | 16.80 | −1.48 | 1.39E−03 |
| 9727 | 14.63 | 14.55 | 16.85 | 16.92 | −1.47 | 3.10E−03 |
| 10801 | 14.70 | 14.69 | 16.90 | 16.92 | −1.47 | 2.21E−03 |
| 10654 | 14.63 | 14.62 | 16.88 | 16.81 | −1.47 | 1.96E−03 |
| 10456 | 14.80 | 14.76 | 17.01 | 17.02 | −1.47 | 1.81E−03 |
| 10711 | 15.03 | 15.19 | 17.17 | 17.12 | −1.46 | 1.55E−03 |
| 9635 | 14.91 | 14.99 | 17.07 | 17.06 | −1.46 | 1.48E−03 |
| 10722 | 14.76 | 14.72 | 16.97 | 16.99 | −1.46 | 2.45E−03 |
| 10277 | 14.55 | 14.55 | 16.77 | 16.73 | −1.46 | 2.17E−03 |
| 10823 | 15.06 | 15.48 | 17.12 | 16.96 | −1.46 | 3.47E−04 |
| 10484 | 14.90 | 15.23 | 16.99 | 16.83 | −1.45 | 1.79E−03 |
| 10636 | 14.82 | 14.85 | 17.00 | 16.96 | −1.45 | 2.05E−03 |
| 10281 | 14.98 | 15.01 | 17.11 | 17.17 | −1.45 | 2.64E−03 |
| 45631 | 14.01 | 14.62 | 15.33 | 16.41 | −1.44 | 3.04E−03 |
| 9843 | 14.84 | 15.02 | 16.95 | 16.89 | −1.44 | 1.15E−03 |
| 9694 | 14.98 | 14.98 | 17.11 | 17.13 | −1.42 | 2.25E−03 |
| 9954 | 14.14 | 13.96 | 16.31 | 16.41 | −1.42 | 5.34E−03 |
| 10347 | 14.59 | 14.65 | 16.68 | 16.67 | −1.41 | 5.55E−03 |
| 10661 | 15.08 | 15.22 | 17.23 | 17.01 | −1.41 | 4.18E−03 |
| 46647 | 14.02 | 14.70 | 15.31 | 16.25 | −1.40 | 5.67E−03 |
| 10243 | 15.16 | 15.25 | 17.23 | 17.19 | −1.40 | 2.27E−03 |
| 10313 | 14.90 | 14.82 | 17.04 | 17.02 | −1.39 | 3.12E−03 |
| 10134 | 14.88 | 14.84 | 17.01 | 16.95 | −1.39 | 3.03E−03 |
| 9884 | 14.88 | 14.84 | 17.01 | 16.95 | −1.39 | 3.03E−03 |
| 10230 | 14.73 | 14.80 | 16.79 | 16.76 | −1.39 | 2.47E−03 |
| 46449 | 14.24 | 14.73 | 15.63 | 16.50 | −1.38 | 4.48E−03 |
| 9763 | 14.76 | 14.98 | 16.73 | 16.70 | −1.38 | 1.61E−03 |
| 9794 | 14.50 | 14.85 | 16.54 | 16.24 | −1.38 | 2.62E−03 |
| 10397 | 13.99 | 14.13 | 16.18 | 15.79 | −1.37 | 1.53E−03 |
| 10750 | 15.25 | 15.35 | 17.30 | 17.22 | −1.37 | 2.58E−03 |
| 10803 | 15.29 | 15.37 | 17.36 | 17.24 | −1.37 | 2.29E−03 |
| 10044 | 15.21 | 15.41 | 17.23 | 17.07 | −1.36 | 3.61E−03 |
| 10561 | 15.21 | 15.41 | 17.23 | 17.07 | −1.36 | 3.61E−03 |
| 10441 | 15.34 | 15.53 | 17.36 | 17.19 | −1.35 | 2.28E−03 |
| 9992 | 15.34 | 15.53 | 17.36 | 17.19 | −1.35 | 2.28E−03 |
| 10675 | 15.34 | 15.53 | 17.36 | 17.19 | −1.35 | 2.28E−03 |
| 9670 | 15.34 | 15.53 | 17.36 | 17.19 | −1.35 | 2.28E−03 |
| 9899 | 15.34 | 15.53 | 17.36 | 17.19 | −1.35 | 2.28E−03 |
| 10116 | 14.08 | 15.17 | 15.79 | 15.30 | −1.35 | 4.57E−03 |
| 10713 | 15.36 | 15.50 | 17.38 | 17.23 | −1.34 | 2.54E−03 |
| 10201 | 15.36 | 15.50 | 17.38 | 17.23 | −1.34 | 2.54E−03 |
| 10074 | 15.36 | 15.50 | 17.38 | 17.23 | −1.34 | 2.54E−03 |
| 10619 | 15.36 | 15.50 | 17.38 | 17.23 | −1.34 | 2.54E−03 |
| 9770 | 15.36 | 15.50 | 17.38 | 17.23 | −1.34 | 2.54E−03 |
| 10660 | 15.36 | 15.50 | 17.38 | 17.23 | −1.34 | 2.54E−03 |
| 9717 | 15.36 | 15.50 | 17.38 | 17.23 | −1.34 | 2.54E−03 |
| 9779 | 15.36 | 15.50 | 17.38 | 17.23 | −1.34 | 2.54E−03 |
| 10680 | 15.36 | 15.50 | 17.38 | 17.23 | −1.34 | 2.54E−03 |
| 10299 | 15.36 | 15.50 | 17.38 | 17.23 | −1.34 | 2.54E−03 |
| 10278 | 15.36 | 15.50 | 17.38 | 17.23 | −1.34 | 2.54E−03 |
| 10640 | 15.36 | 15.50 | 17.38 | 17.23 | −1.34 | 2.54E−03 |
| 10634 | 15.36 | 15.50 | 17.38 | 17.23 | −1.34 | 2.54E−03 |
| 9714 | 15.36 | 15.50 | 17.38 | 17.23 | −1.34 | 2.54E−03 |
| 10418 | 15.36 | 15.50 | 17.38 | 17.23 | −1.34 | 2.54E−03 |
| 10354 | 15.36 | 15.50 | 17.38 | 17.23 | −1.34 | 2.54E−03 |
| 10216 | 15.36 | 15.50 | 17.38 | 17.23 | −1.34 | 2.54E−03 |
| 10519 | 15.36 | 15.50 | 17.38 | 17.23 | −1.34 | 2.54E−03 |
| 9806 | 15.36 | 15.50 | 17.38 | 17.23 | −1.34 | 2.54E−03 |
| 9977 | 15.36 | 15.50 | 17.38 | 17.23 | −1.34 | 2.54E−03 |
| 10420 | 15.36 | 15.50 | 17.38 | 17.23 | −1.34 | 2.54E−03 |
| 10403 | 15.36 | 15.50 | 17.38 | 17.23 | −1.34 | 2.54E−03 |
| 10287 | 15.05 | 15.10 | 17.06 | 17.01 | −1.34 | 3.58E−03 |
| 10246 | 14.96 | 14.81 | 17.03 | 17.03 | −1.33 | 4.27E−03 |
| 9981 | 15.19 | 15.23 | 17.23 | 17.11 | −1.33 | 2.55E−03 |
| 10301 | 15.46 | 15.63 | 17.46 | 17.29 | −1.33 | 2.54E−03 |
| 10386 | 14.91 | 14.98 | 16.92 | 16.81 | −1.33 | 3.00E−03 |
| 9741 | 15.29 | 15.43 | 17.26 | 17.14 | −1.32 | 2.25E−03 |
| 10436 | 13.65 | 14.21 | 15.42 | 15.26 | −1.31 | 5.82E−03 |
| 10317 | 15.34 | 15.51 | 17.31 | 17.12 | −1.31 | 2.50E−03 |
| 10372 | 15.24 | 15.35 | 17.21 | 17.07 | −1.30 | 3.69E−03 |
| 10407 | 15.01 | 15.21 | 16.99 | 16.73 | −1.30 | 1.63E−03 |
| 9814 | 14.75 | 14.89 | 16.70 | 16.53 | −1.29 | 5.05E−03 |
| 10829 | 14.93 | 14.87 | 16.95 | 16.85 | −1.29 | 4.03E−03 |
| 9919 | 14.98 | 15.39 | 16.88 | 16.53 | −1.29 | 1.25E−03 |
| 10736 | 15.43 | 15.59 | 17.37 | 17.18 | −1.29 | 2.44E−03 |
| 10624 | 15.42 | 15.62 | 17.36 | 17.14 | −1.28 | 2.23E−03 |
| 10504 | 14.92 | 14.83 | 16.94 | 16.85 | −1.28 | 4.69E−03 |
| 9660 | 15.42 | 15.55 | 17.34 | 17.17 | −1.27 | 2.66E−03 |
| 10559 | 15.34 | 15.50 | 17.26 | 17.05 | −1.27 | 2.63E−03 |
| 9667 | 15.42 | 15.52 | 17.36 | 17.15 | −1.26 | 3.01E−03 |
| 10026 | 15.00 | 15.21 | 16.95 | 16.62 | −1.26 | 3.21E−03 |
| 10344 | 14.71 | 14.85 | 16.69 | 16.37 | −1.26 | 4.04E−03 |
| 9993 | 15.49 | 15.70 | 17.39 | 17.12 | −1.25 | 1.20E−03 |
| 10235 | 15.35 | 15.38 | 17.31 | 17.11 | −1.25 | 5.34E−03 |
| 10324 | 14.90 | 15.01 | 16.81 | 16.63 | −1.25 | 4.21E−03 |
| 10767 | 15.07 | 15.39 | 16.94 | 16.60 | −1.24 | 3.90E−03 |
| 9812 | 15.46 | 15.69 | 17.32 | 17.09 | −1.24 | 3.14E−03 |
| 10718 | 14.74 | 14.64 | 16.66 | 16.62 | −1.24 | 5.96E−03 |
| 45563 | 14.46 | 15.03 | 15.84 | 16.23 | −1.24 | 5.23E−03 |
| 10495 | 15.63 | 15.83 | 17.50 | 17.23 | −1.23 | 3.88E−03 |
| 10743 | 14.86 | 14.99 | 16.76 | 16.49 | −1.22 | 3.62E−03 |
| 9905 | 14.04 | 14.67 | 15.61 | 15.48 | −1.22 | 6.65E−04 |
| 10703 | 13.72 | 14.28 | 15.37 | 15.16 | −1.21 | 5.13E−03 |
| 9927 | 15.66 | 15.79 | 17.55 | 17.26 | −1.21 | 4.30E−03 |
| 9790 | 15.70 | 15.86 | 17.57 | 17.27 | −1.20 | 5.02E−03 |
| 9706 | 15.70 | 15.86 | 17.57 | 17.27 | −1.20 | 5.02E−03 |
| 10721 | 15.23 | 15.45 | 17.02 | 16.70 | −1.17 | 3.77E−03 |
| 10258 | 15.58 | 15.85 | 17.35 | 17.02 | −1.16 | 4.27E−03 |
| 10385 | 15.62 | 16.05 | 17.35 | 16.89 | −1.14 | 4.11E−03 |
| 9847 | 15.29 | 15.56 | 17.03 | 16.65 | −1.12 | 5.11E−03 |
| 10781 | 15.67 | 15.89 | 17.40 | 17.08 | −1.12 | 4.95E−03 |
| 10202 | 14.17 | 14.71 | 15.70 | 15.38 | −1.09 | 3.47E−03 |

TABLE 2
Taxonomy corresponding to OTUs increased following irradiation

| OTU | Full Taxonomy |
|---|---|
| 31902 | Bacteria:Cyanobacteria:YS2_SP:Rs-H34_CL:Unclassified:Unclassified:sfA |
| 46156 | Bacteria:Bacteroidetes:p-184-o5_SP:C9_B12_CL:Bacteroidales:Unclassified:sfA |
| 31674 | Bacteria:Cyanobacteria:YS2_SP:Rs-H34_CL:Unclassified:Unclassified:sfA |
| 46174 | Bacteria:Bacteroidetes:p-184-o5_SP:Bacteroidales_CL:Bacteroidales:Unclassified:sfA |
| 45878 | Bacteria:Bacteroidetes:p-184-o5_SP:Bacteroidales_CL:Bacteroidales:Unclassified:sfA |
| 46045 | Bacteria:Bacteroidetes:p-184-o5_SP:Bacteroidales_CL:Bacteroidales:Unclassified:sfK |
| 46464 | Bacteria:Bacteroidetes:p-184-o5_SP:Bacteroidales_CL:Bacteroidales:Unclassified:sfA |
| 46118 | Bacteria:Bacteroidetes:p-184-o5_SP:Bacteroidales_CL:Bacteroidales:Unclassified:sfA |
| 46123 | Bacteria:Bacteroidetes:p-184-o5_SP:Bacteroidales_CL:Bacteroidales:Unclassified:sfA |
| 10509 | Bacteria:Firmicutes:Bacilli_SP:Lactobacillales_CL:Lactobacillales:Lactobacillaceae:sfA |
| 10461 | Bacteria:Firmicutes:Bacilli_SP:Lactobacillales_CL:Lactobacillales:Lactobacillaceae:sfA |
| 46422 | Bacteria:Bacteroidetes:p-184-o5_SP:Bacteroidales_CL:Bacteroidales:Unclassified:sfA |
| 10021 | Bacteria:Firmicutes:Bacilli_SP:Lactobacillales_CL:Lactobacillales:Lactobacillaceae:sfA |
| 46592 | Bacteria:Bacteroidetes:p-184-o5_SP:Bacteroidales_CL:Bacteroidales:Unclassified:sfA |
| 45539 | Bacteria:Bacteroidetes:p-184-o5_SP:rc5-47_CL:Bacteroidales:Unclassified:sfA |
| 46370 | Bacteria:Bacteroidetes:p-184-o5_SP:SWPT15_aaa02b04_CL:Bacteroidales:Unclassified:sfA |
| 9685 | Bacteria:Firmicutes:Bacilli_SP:Lactobacillales_CL:Lactobacillales:Lactobacillaceae:sfA |
| 46656 | Bacteria:Bacteroidetes:p-184-o5_SP:Bacteroidales_CL:Bacteroidales:Unclassified:sfA |
| 10405 | Bacteria:Firmicutes:Bacilli_SP:Lactobacillales_CL:Lactobacillales:Lactobacillaceae:sfA |
| 10597 | Bacteria:Firmicutes:Bacilli_SP:Lactobacillales_CL:Lactobacillales:Lactobacillaceae:sfA |
| 10577 | Bacteria:Firmicutes:Bacilli_SP:Lactobacillales_CL:Lactobacillales:Lactobacillaceae:sfA |
| 10309 | Bacteria:Firmicutes:Bacilli_SP:Lactobacillales_CL:Lactobacillales:Lactobacillaceae:sfA |
| 10155 | Bacteria:Firmicutes:Bacilli_SP:Lactobacillales_CL:Lactobacillales:Lactobacillaceae:sfA |
| 10849 | Bacteria:Firmicutes:Bacilli_SP:Lactobacillales_CL:Lactobacillales:Lactobacillaceae:sfA |
| 45635 | Bacteria:Bacteroidetes:p-184-o5_SP:rc2-15_CL:Bacteroidales:Unclassified:sfA |
| 10632 | Bacteria:Firmicutes:Bacilli_SP:Lactobacillales_CL:Lactobacillales:Lactobacillaceae:sfA |
| 32070 | Bacteria:Cyanobacteria:YS2_SP:Rs-H34_CL:Unclassified:Unclassified:sfA |
| 46404 | Bacteria:Bacteroidetes:p-184-o5_SP:Bacteroidales_CL:Bacteroidales:Unclassified:sfA |
| 46326 | Bacteria:Bacteroidetes:p-184-o5_SP:Bacteroidales_CL:Bacteroidales:Unclassified:sfA |
| 46044 | Bacteria:Bacteroidetes:p-184-o5_SP:Bacteroidales_CL:Bacteroidales:Unclassified:sfA |
| 10168 | Bacteria:Firmicutes:Bacilli_SP:Lactobacillales_CL:Lactobacillales:Lactobacillaceae:sfA |
| 46556 | Bacteria:Bacteroidetes:p-184-o5_SP:rc5-47_CL:Bacteroidales:Unclassified:sfA |
| 10516 | Bacteria:Firmicutes:Bacilli_SP:Lactobacillales_CL:Lactobacillales:Lactobacillaceae:sfA |
| 9808 | Bacteria:Firmicutes:Bacilli_SP:Lactobacillales_CL:Lactobacillales:Lactobacillaceae:sfA |
| 10830 | Bacteria:Firmicutes:Bacilli_SP:Lactobacillales_CL:Lactobacillales:Lactobacillaceae:sfA |
| 10768 | Bacteria:Firmicutes:Bacilli_SP:Lactobacillales_CL:Lactobacillales:Streptococcaceae:sfA |
| 10001 | Bacteria:Firmicutes:Bacilli_SP:Lactobacillales_CL:Lactobacillales:Lactobacillaceae:sfA |
| 45933 | Bacteria:Bacteroidetes:p-184-o5_SP:rc5-47_CL:Bacteroidales:Unclassified:sfA |
| 10232 | Bacteria:Firmicutes:Bacilli_SP:Lactobacillales_CL:Lactobacillales:Streptococcaceae:sfA |
| 10250 | Bacteria:Firmicutes:Bacilli_SP:Lactobacillales_CL:Lactobacillales:Streptococcaceae:sfA |
| 10596 | Bacteria:Firmicutes:Bacilli_SP:Lactobacillales_CL:Lactobacillales:Streptococcaceae:sfA |
| 35067 | Bacteria:Firmicutes:Clostridia_SP:C18_h03_2_CL:Clostridiales:Unclassified:sfA |
| 9680 | Bacteria:Firmicutes:Bacilli_SP:Lactobacillales_CL:Lactobacillales:Lactobacillaceae:sfA |
| 10858 | Bacteria:Firmicutes:Bacilli_SP:Lactobacillales_CL:Lactobacillales:Lactobacillaceae:sfA |
| 10122 | Bacteria:Firmicutes:Bacilli_SP:Lactobacillales_CL:Lactobacillales:Lactobacillaceae:sfA |
| 10078 | Bacteria:Firmicutes:Bacilli_SP:Lactobacillales_CL:Lactobacillales:Lactobacillaceae:sfA |
| 9710 | Bacteria:Firmicutes:Bacilli_SP:Lactobacillales_CL:Lactobacillales:Lactobacillaceae:sfA |
| 31568 | Bacteria:Cyanobacteria:YS2_SP:Rs-H34_CL:Unclassified:Unclassified:sfA |
| 45735 | Bacteria:Bacteroidetes:p-184-o5_SP:Bacteroidales_CL:Bacteroidales:Unclassified:sfA |
| 10520 | Bacteria:Firmicutes:Bacilli_SP:Lactobacillales_CL:Lactobacillales:Lactobacillaceae:sfA |
| 10148 | Bacteria:Firmicutes:Bacilli_SP:Lactobacillales_CL:Lactobacillales:Lactobacillaceae:sfA |
| 9839 | Bacteria:Firmicutes:Bacilli_SP:Lactobacillales_CL:Lactobacillales:Streptococcaceae:sfA |
| 31504 | Bacteria:Cyanobacteria:YS2_SP:Rs-H34_CL:Unclassified:Unclassified:sfA |
| 45848 | Bacteria:Bacteroidetes:p-184-o5_SP:Bacteroidales_CL:Bacteroidales:Unclassified:sfG |
| 45505 | Bacteria:Bacteroidetes:p-184-o5_SP:Bacteroidales_CL:Bacteroidales:Unclassified:sfG |
| 46054 | Bacteria:Bacteroidetes:p-184-o5_SP:Bacteroidales_CL:Bacteroidales:Unclassified:sfG |
| 10701 | Bacteria:Firmicutes:Bacilli_SP:Lactobacillales_CL:Lactobacillales:Lactobacillaceae:sfA |
| 10474 | Bacteria:Firmicutes:Bacilli_SP:Lactobacillales_CL:Lactobacillales:Lactobacillaceae:sfA |
| 10135 | Bacteria:Firmicutes:Bacilli_SP:Lactobacillales_CL:Lactobacillales:Lactobacillaceae:sfA |
| 10356 | Bacteria:Firmicutes:Bacilli_SP:Lactobacillales_CL:Lactobacillales:Lactobacillaceae:sfA |
| 9747 | Bacteria:Firmicutes:Bacilli_SP:Lactobacillales_CL:Lactobacillales:Lactobacillaceae:sfA |
| 9883 | Bacteria:Firmicutes:Bacilli_SP:Lactobacillales_CL:Lactobacillales:Lactobacillaceae:sfA |
| 10017 | Bacteria:Firmicutes:Bacilli_SP:Lactobacillales_CL:Lactobacillales:Lactobacillaceae:sfA |
| 10140 | Bacteria:Firmicutes:Bacilli_SP:Lactobacillales_CL:Lactobacillales:Lactobacillaceae:sfA |
| 9727 | Bacteria:Firmicutes:Bacilli_SP:Lactobacillales_CL:Lactobacillales:Lactobacillaceae:sfA |
| 10801 | Bacteria:Firmicutes:Bacilli_SP:Lactobacillales_CL:Lactobacillales:Lactobacillaceae:sfA |
| 10654 | Bacteria:Firmicutes:Bacilli_SP:Lactobacillales_CL:Lactobacillales:Lactobacillaceae:sfA |
| 10456 | Bacteria:Firmicutes:Bacilli_SP:Lactobacillales_CL:Lactobacillales:Lactobacillaceae:sfA |
| 10711 | Bacteria:Firmicutes:Bacilli_SP:Lactobacillales_CL:Lactobacillales:Lactobacillaceae:sfA |
| 9635 | Bacteria:Firmicutes:Bacilli_SP:Lactobacillales_CL:Lactobacillales:Lactobacillaceae:sfA |
| 10722 | Bacteria:Firmicutes:Bacilli_SP:Lactobacillales_CL:Lactobacillales:Lactobacillaceae:sfA |
| 10277 | Bacteria:Firmicutes:Bacilli_SP:Lactobacillales_CL:Lactobacillales:Lactobacillaceae:sfA |
| 10823 | Bacteria:Firmicutes:Bacilli_SP:Lactobacillales_CL:Lactobacillales:Lactobacillaceae:sfA |
| 10484 | Bacteria:Firmicutes:Bacilli_SP:Lactobacillales_CL:Lactobacillales:Lactobacillaceae:sfA |
| 10636 | Bacteria:Firmicutes:Bacilli_SP:Lactobacillales_CL:Lactobacillales:Lactobacillaceae:sfA |
| 10281 | Bacteria:Firmicutes:Bacilli_SP:Lactobacillales_CL:Lactobacillales:Lactobacillaceae:sfA |
| 45631 | Bacteria:Bacteroidetes:p-184-o5_SP:rc5-47_CL:Bacteroidales:Unclassified:sfA |
| 9843 | Bacteria:Firmicutes:Bacilli_SP:Lactobacillales_CL:Lactobacillales:Lactobacillaceae:sfA |
| 9694 | Bacteria:Firmicutes:Bacilli_SP:Lactobacillales_CL:Lactobacillales:Lactobacillaceae:sfA |
| 9954 | Bacteria:Firmicutes:Bacilli_SP:Lactobacillales_CL:Lactobacillales:Lactobacillaceae:sfA |
| 10347 | Bacteria:Firmicutes:Bacilli_SP:Lactobacillales_CL:Lactobacillales:Lactobacillaceae:sfA |
| 10661 | Bacteria:Firmicutes:Bacilli_SP:Lactobacillales_CL:Lactobacillales:Lactobacillaceae:sfA |
| 46647 | Bacteria:Bacteroidetes:p-184-o5_SP:Bacteroidales_CL:Bacteroidales:Unclassified:sfG |
| 10243 | Bacteria:Firmicutes:Bacilli_SP:Lactobacillales_CL:Lactobacillales:Lactobacillaceae:sfA |
| 10313 | Bacteria:Firmicutes:Bacilli_SP:Lactobacillales_CL:Lactobacillales:Lactobacillaceae:sfA |
| 10134 | Bacteria:Firmicutes:Bacilli_SP:Lactobacillales_CL:Lactobacillales:Lactobacillaceae:sfA |
| 9884 | Bacteria:Firmicutes:Bacilli_SP:Lactobacillales_CL:Lactobacillales:Lactobacillaceae:sfA |
| 10230 | Bacteria:Firmicutes:Bacilli_SP:Lactobacillales_CL:Lactobacillales:Lactobacillaceae:sfA |
| 46449 | Bacteria:Bacteroidetes:p-184-o5_SP:Bacteroidales_CL:Bacteroidales:Unclassified:sfA |
| 9763 | Bacteria:Firmicutes:Bacilli_SP:Lactobacillales_CL:Lactobacillales:Lactobacillaceae:sfA |
| 9794 | Bacteria:Firmicutes:Bacilli_SP:Lactobacillales_CL:Lactobacillales:Lactobacillaceae:sfA |
| 10397 | Bacteria:Firmicutes:Bacilli_SP:Lactobacillales_CL:Lactobacillales:Lactobacillaceae:sfA |
| 10750 | Bacteria:Firmicutes:Bacilli_SP:Lactobacillales_CL:Lactobacillales:Lactobacillaceae:sfA |
| 10803 | Bacteria:Firmicutes:Bacilli_SP:Lactobacillales_CL:Lactobacillales:Lactobacillaceae:sfA |
| 10044 | Bacteria:Firmicutes:Bacilli_SP:Lactobacillales_CL:Lactobacillales:Lactobacillaceae:sfA |
| 10561 | Bacteria:Firmicutes:Bacilli_SP:Lactobacillales_CL:Lactobacillales:Lactobacillaceae:sfA |
| 10441 | Bacteria:Firmicutes:Bacilli_SP:Lactobacillales_CL:Lactobacillales:Lactobacillaceae:sfA |
| 9992 | Bacteria:Firmicutes:Bacilli_SP:Lactobacillales_CL:Lactobacillales:Lactobacillaceae:sfA |
| 10675 | Bacteria:Firmicutes:Bacilli_SP:Lactobacillales_CL:Lactobacillales:Lactobacillaceae:sfA |
| 9670 | Bacteria:Firmicutes:Bacilli_SP:Lactobacillales_CL:Lactobacillales:Lactobacillaceae:sfA |
| 9899 | Bacteria:Firmicutes:Bacilli_SP:Lactobacillales_CL:Lactobacillales:Lactobacillaceae:sfA |
| 10116 | Bacteria:Firmicutes:Bacilli_SP:Lactobacillales_CL:Lactobacillales:Streptococcaceae:sfA |
| 10713 | Bacteria:Firmicutes:Bacilli_SP:Lactobacillales_CL:Lactobacillales:Lactobacillaceae:sfA |
| 10201 | Bacteria:Firmicutes:Bacilli_SP:Lactobacillales_CL:Lactobacillales:Lactobacillaceae:sfA |
| 10074 | Bacteria:Firmicutes:Bacilli_SP:Lactobacillales_CL:Lactobacillales:Lactobacillaceae:sfA |
| 10619 | Bacteria:Firmicutes:Bacilli_SP:Lactobacillales_CL:Lactobacillales:Lactobacillaceae:sfA |
| 9770 | Bacteria:Firmicutes:Bacilli_SP:Lactobacillales_CL:Lactobacillales:Lactobacillaceae:sfA |
| 10660 | Bacteria:Firmicutes:Bacilli_SP:Lactobacillales_CL:Lactobacillales:Lactobacillaceae:sfA |
| 9717 | Bacteria:Firmicutes:Bacilli_SP:Lactobacillales_CL:Lactobacillales:Lactobacillaceae:sfA |
| 9779 | Bacteria:Firmicutes:Bacilli_SP:Lactobacillales_CL:Lactobacillales:Lactobacillaceae:sfA |
| 10680 | Bacteria:Firmicutes:Bacilli_SP:Lactobacillales_CL:Lactobacillales:Lactobacillaceae:sfA |
| 10299 | Bacteria:Firmicutes:Bacilli_SP:Lactobacillales_CL:Lactobacillales:Lactobacillaceae:sfA |
| 10278 | Bacteria:Firmicutes:Bacilli_SP:Lactobacillales_CL:Lactobacillales:Lactobacillaceae:sfA |
| 10640 | Bacteria:Firmicutes:Bacilli_SP:Lactobacillales_CL:Lactobacillales:Lactobacillaceae:sfA |
| 10634 | Bacteria:Firmicutes:Bacilli_SP:Lactobacillales_CL:Lactobacillales:Lactobacillaceae:sfA |
| 9714 | Bacteria:Firmicutes:Bacilli_SP:Lactobacillales_CL:Lactobacillales:Lactobacillaceae:sfA |
| 10418 | Bacteria:Firmicutes:Bacilli_SP:Lactobacillales_CL:Lactobacillales:Lactobacillaceae:sfA |
| 10354 | Bacteria:Firmicutes:Bacilli_SP:Lactobacillales_CL:Lactobacillales:Lactobacillaceae:sfA |
| 10216 | Bacteria:Firmicutes:Bacilli_SP:Lactobacillales_CL:Lactobacillales:Lactobacillaceae:sfA |
| 10519 | Bacteria:Firmicutes:Bacilli_SP:Lactobacillales_CL:Lactobacillales:Lactobacillaceae:sfA |
| 9806 | Bacteria:Firmicutes:Bacilli_SP:Lactobacillales_CL:Lactobacillales:Lactobacillaceae:sfA |
| 9977 | Bacteria:Firmicutes:Bacilli_SP:Lactobacillales_CL:Lactobacillales:Lactobacillaceae:sfA |
| 10420 | Bacteria:Firmicutes:Bacilli_SP:Lactobacillales_CL:Lactobacillales:Lactobacillaceae:sfA |
| 10403 | Bacteria:Firmicutes:Bacilli_SP:Lactobacillales_CL:Lactobacillales:Lactobacillaceae:sfA |
| 10287 | Bacteria:Firmicutes:Bacilli_SP:Lactobacillales_CL:Lactobacillales:Lactobacillaceae:sfA |
| 10246 | Bacteria:Firmicutes:Bacilli_SP:Lactobacillales_CL:Lactobacillales:Lactobacillaceae:sfA |
| 9981 | Bacteria:Firmicutes:Bacilli_SP:Lactobacillales_CL:Lactobacillales:Lactobacillaceae:sfA |
| 10301 | Bacteria:Firmicutes:Bacilli_SP:Lactobacillales_CL:Lactobacillales:Lactobacillaceae:sfA |
| 10386 | Bacteria:Firmicutes:Bacilli_SP:Lactobacillales_CL:Lactobacillales:Lactobacillaceae:sfA |
| 9741 | Bacteria:Firmicutes:Bacilli_SP:Lactobacillales_CL:Lactobacillales:Lactobacillaceae:sfA |
| 10436 | Bacteria:Firmicutes:Bacilli_SP:Lactobacillales_CL:Lactobacillales:Streptococcaceae:sfA |
| 10317 | Bacteria:Firmicutes:Bacilli_SP:Lactobacillales_CL:Lactobacillales:Lactobacillaceae:sfA |
| 10372 | Bacteria:Firmicutes:Bacilli_SP:Lactobacillales_CL:Lactobacillales:Lactobacillaceae:sfA |
| 10407 | Bacteria:Firmicutes:Bacilli_SP:Lactobacillales_CL:Lactobacillales:Lactobacillaceae:sfA |
| 9814 | Bacteria:Firmicutes:Bacilli_SP:Lactobacillales_CL:Lactobacillales:Lactobacillaceae:sfA |
| 10829 | Bacteria:Firmicutes:Bacilli_SP:Lactobacillales_CL:Lactobacillales:Lactobacillaceae:sfA |
| 9919 | Bacteria:Firmicutes:Bacilli_SP:Lactobacillales_CL:Lactobacillales:Lactobacillaceae:sfA |
| 10736 | Bacteria:Firmicutes:Bacilli_SP:Lactobacillales_CL:Lactobacillales:Lactobacillaceae:sfA |
| 10624 | Bacteria:Firmicutes:Bacilli_SP:Lactobacillales_CL:Lactobacillales:Lactobacillaceae:sfA |
| 10504 | Bacteria:Firmicutes:Bacilli_SP:Lactobacillales_CL:Lactobacillales:Lactobacillaceae:sfA |
| 9660 | Bacteria:Firmicutes:Bacilli_SP:Lactobacillales_CL:Lactobacillales:Lactobacillaceae:sfA |
| 10559 | Bacteria:Firmicutes:Bacilli_SP:Lactobacillales_CL:Lactobacillales:Lactobacillaceae:sfA |
| 9667 | Bacteria:Firmicutes:Bacilli_SP:Lactobacillales_CL:Lactobacillales:Lactobacillaceae:sfA |
| 10026 | Bacteria:Firmicutes:Bacilli_SP:Lactobacillales_CL:Lactobacillales:Lactobacillaceae:sfA |
| 10344 | Bacteria:Firmicutes:Bacilli_SP:Lactobacillales_CL:Lactobacillales:Lactobacillaceae:sfA |
| 9993 | Bacteria:Firmicutes:Bacilli_SP:Lactobacillales_CL:Lactobacillales:Lactobacillaceae:sfA |
| 10235 | Bacteria:Firmicutes:Bacilli_SP:Lactobacillales_CL:Lactobacillales:Lactobacillaceae:sfA |
| 10324 | Bacteria:Firmicutes:Bacilli_SP:Lactobacillales_CL:Lactobacillales:Lactobacillaceae:sfA |
| 10767 | Bacteria:Firmicutes:Bacilli_SP:Lactobacillales_CL:Lactobacillales:Lactobacillaceae:sfA |
| 9812 | Bacteria:Firmicutes:Bacilli_SP:Lactobacillales_CL:Lactobacillales:Lactobacillaceae:sfA |
| 10718 | Bacteria:Firmicutes:Bacilli_SP:Lactobacillales_CL:Lactobacillales:Lactobacillaceae:sfA |
| 45563 | Bacteria:Bacteroidetes:p-184-o5_SP:Bacteroidales_CL:Bacteroidales:Unclassified:sfA |
| 10495 | Bacteria:Firmicutes:Bacilli_SP:Lactobacillales_CL:Lactobacillales:Lactobacillaceae:sfA |
| 10743 | Bacteria:Firmicutes:Bacilli_SP:Lactobacillales_CL:Lactobacillales:Lactobacillaceae:sfA |
| 9905 | Bacteria:Firmicutes:Bacilli_SP:Lactobacillales_CL:Lactobacillales:Streptococcaceae:sfA |
| 10703 | Bacteria:Firmicutes:Bacilli_SP:Lactobacillales_CL:Lactobacillales:Streptococcaceae:sfA |
| 9927 | Bacteria:Firmicutes:Bacilli_SP:Lactobacillales_CL:Lactobacillales:Lactobacillaceae:sfA |
| 9790 | Bacteria:Firmicutes:Bacilli_SP:Lactobacillales_CL:Lactobacillales:Lactobacillaceae:sfA |
| 9706 | Bacteria:Firmicutes:Bacilli_SP:Lactobacillales_CL:Lactobacillales:Lactobacillaceae:sfA |
| 10721 | Bacteria:Firmicutes:Bacilli_SP:Lactobacillales_CL:Lactobacillales:Lactobacillaceae:sfA |
| 10258 | Bacteria:Firmicutes:Bacilli_SP:Lactobacillales_CL:Lactobacillales:Lactobacillaceae:sfA |
| 10385 | Bacteria:Firmicutes:Bacilli_SP:Lactobacillales_CL:Lactobacillales:Lactobacillaceae:sfA |
| 9847 | Bacteria:Firmicutes:Bacilli_SP:Lactobacillales_CL:Lactobacillales:Lactobacillaceae:sfA |
| 10781 | Bacteria:Firmicutes:Bacilli_SP:Lactobacillales_CL:Lactobacillales:Lactobacillaceae:sfA |
| 10202 | Bacteria:Firmicutes:Bacilli_SP:Lactobacillales_CL:Lactobacillales:Streptococcaceae:sfA |

TABLE 3
OTUs decreased following irradiation
Log2 Fold
Change

| | Day Averaged Log2 | | | | | |
|---|---|---|---|---|---|---|
| | Signal Values | | | Pre vs. Post Radiation | | |
| OTU | 0 | 4 | 11 | 21 | Log2 | p-value |
| 39143 | 15.33 | 14.40 | 13.04 | 12.71 | 1.94 | 1.88E−03 |
| 39672 | 15.28 | 14.33 | 13.30 | 12.72 | 1.83 | 2.35E−03 |
| 39286 | 15.43 | 14.56 | 13.15 | 12.80 | 1.92 | 2.37E−03 |
| 39546 | 15.20 | 14.39 | 12.94 | 12.59 | 1.90 | 2.49E−03 |
| 39988 | 15.34 | 14.55 | 13.31 | 12.93 | 1.74 | 3.49E−03 |
| 39172 | 15.33 | 14.47 | 13.51 | 12.97 | 1.68 | 3.51E−03 |
| 39360 | 15.55 | 14.74 | 13.66 | 13.13 | 1.71 | 3.66E−03 |
| 39375 | 15.41 | 14.59 | 13.60 | 13.09 | 1.65 | 3.66E−03 |
| 39292 | 15.25 | 14.35 | 13.42 | 12.87 | 1.71 | 3.89E−03 |
| 40032 | 15.25 | 14.35 | 13.42 | 12.87 | 1.71 | 3.89E−03 |
| 38998 | 15.35 | 14.53 | 13.61 | 13.04 | 1.62 | 4.54E−03 |
| 39482 | 15.41 | 14.51 | 13.86 | 13.17 | 1.57 | 4.75E−03 |
| 40092 | 15.24 | 14.46 | 13.41 | 12.96 | 1.63 | 4.98E−03 |
| 39490 | 15.42 | 14.64 | 13.83 | 13.24 | 1.52 | 5.42E−03 |
| 39320 | 15.45 | 14.66 | 13.86 | 13.28 | 1.51 | 5.67E−03 |
| 39540 | 15.29 | 14.42 | 13.71 | 13.08 | 1.55 | 5.72E−03 |
| 39162 | 15.30 | 14.42 | 13.72 | 13.08 | 1.56 | 5.72E−03 |
| 39354 | 15.23 | 14.39 | 13.56 | 12.97 | 1.58 | 5.88E−03 |
| 39536 | 15.18 | 14.32 | 13.52 | 12.99 | 1.57 | 5.95E−03 |
| 39645 | 15.24 | 14.38 | 13.80 | 13.16 | 1.46 | 6.01E−03 |
| 40015 | 15.35 | 14.59 | 13.73 | 13.14 | 1.53 | 6.16E−03 |
| 39762 | 15.27 | 14.57 | 13.57 | 13.17 | 1.50 | 6.58E−03 |
| 38995 | 15.35 | 14.61 | 13.79 | 13.23 | 1.47 | 7.30E−03 |
| 40033 | 15.39 | 14.66 | 13.92 | 13.33 | 1.42 | 7.69E−03 |
| 39297 | 15.39 | 14.70 | 13.77 | 13.28 | 1.48 | 7.92E−03 |
| 39153 | 14.89 | 13.95 | 13.45 | 12.86 | 1.47 | 8.04E−03 |
| 39310 | 15.61 | 14.82 | 14.36 | 13.64 | 1.33 | 8.31E−03 |
| 39947 | 15.28 | 14.56 | 13.83 | 13.26 | 1.40 | 8.34E−03 |
| 39741 | 15.43 | 14.58 | 14.09 | 13.52 | 1.37 | 8.39E−03 |
| 39381 | 15.44 | 14.62 | 14.14 | 13.55 | 1.34 | 8.97E−03 |
| 39164 | 15.44 | 14.76 | 14.03 | 13.35 | 1.39 | 8.98E−03 |
| 39283 | 15.34 | 14.65 | 13.96 | 13.33 | 1.36 | 9.27E−03 |
| 39682 | 15.10 | 14.33 | 13.74 | 13.04 | 1.39 | 1.04E−02 |
| 39895 | 15.45 | 14.69 | 14.18 | 13.54 | 1.32 | 1.09E−02 |
| 39747 | 15.31 | 14.56 | 13.96 | 13.21 | 1.40 | 1.12E−02 |
| 39368 | 15.20 | 14.52 | 13.83 | 13.21 | 1.35 | 1.20E−02 |
| 39237 | 15.21 | 14.45 | 13.97 | 13.33 | 1.29 | 1.25E−02 |
| 39814 | 15.12 | 14.39 | 14.07 | 13.30 | 1.20 | 1.30E−02 |
| 39828 | 15.49 | 14.72 | 14.36 | 13.74 | 1.22 | 1.32E−02 |
| 39823 | 15.34 | 14.59 | 14.10 | 13.60 | 1.24 | 1.37E−02 |
| 40020 | 14.96 | 14.13 | 13.80 | 13.11 | 1.27 | 1.39E−02 |
| 40029 | 15.53 | 14.77 | 14.48 | 13.84 | 1.17 | 1.61E−02 |
| 39783 | 15.57 | 14.84 | 14.47 | 13.98 | 1.13 | 1.70E−02 |
| 39870 | 15.28 | 14.49 | 14.20 | 13.62 | 1.18 | 1.95E−02 |
| 39035 | 15.06 | 14.21 | 13.97 | 13.58 | 1.14 | 2.06E−02 |
| 39763 | 15.22 | 14.57 | 14.21 | 13.41 | 1.16 | 2.06E−02 |
| 39475 | 15.43 | 14.79 | 14.51 | 13.83 | 1.05 | 2.11E−02 |

TABLE 4
Taxonomy corresponding to OTUs decreased following irradiation

| OTU | Full Taxonomy |
|---|---|
| 39143 | Bacteria:Firmicutes:Clostridia_SP:Clostridiales_CL:Clostridiales:Clostridiaceae:sfA |
| 39672 | Bacteria:Firmicutes:Clostridia_SP:Clostridiales_CL:Clostridiales:Clostridiaceae:sfA |
| 39286 | Bacteria:Firmicutes:Clostridia_SP:Clostridiales_CL:Clostridiales:Clostridiaceae:sfA |
| 39546 | Bacteria:Firmicutes:Clostridia_SP:Clostridiales_CL:Clostridiales:Clostridiaceae:sfA |
| 39988 | Bacteria:Firmicutes:Clostridia_SP:Clostridiales_CL:Clostridiales:Clostridiaceae:sfA |
| 39172 | Bacteria:Firmicutes:Clostridia_SP:Clostridiales_CL:Clostridiales:Clostridiaceae:sfA |
| 39360 | Bacteria:Firmicutes:Clostridia_SP:Clostridiales_CL:Clostridiales:Clostridiaceae:sfA |
| 39375 | Bacteria:Firmicutes:Clostridia_SP:Clostridiales_CL:Clostridiales:Clostridiaceae:sfA |
| 39292 | Bacteria:Firmicutes:Clostridia_SP:Clostridiales_CL:Clostridiales:Clostridiaceae:sfA |
| 40032 | Bacteria:Firmicutes:Clostridia_SP:Clostridiales_CL:Clostridiales:Clostridiaceae:sfA |
| 38998 | Bacteria:Firmicutes:Clostridia_SP:Clostridiales_CL:Clostridiales:Clostridiaceae:sfA |
| 39482 | Bacteria:Firmicutes:Clostridia_SP:Clostridiales_CL:Clostridiales:Clostridiaceae:sfA |
| 40092 | Bacteria:Firmicutes:Clostridia_SP:Clostridiales_CL:Clostridiales:Clostridiaceae:sfA |
| 39490 | Bacteria:Firmicutes:Clostridia_SP:Clostridiales_CL:Clostridiales:Clostridiaceae:sfA |
| 39320 | Bacteria:Firmicutes:Clostridia_SP:Clostridiales_CL:Clostridiales:Clostridiaceae:sfA |
| 39540 | Bacteria:Firmicutes:Clostridia_SP:Clostridiales_CL:Clostridiales:Clostridiaceae:sfA |
| 39162 | Bacteria:Firmicutes:Clostridia_SP:Clostridiales_CL:Clostridiales:Clostridiaceae:sfA |
| 39354 | Bacteria:Firmicutes:Clostridia_SP:Clostridiales_CL:Clostridiales:Clostridiaceae:sfA |
| 39536 | Bacteria:Firmicutes:Clostridia_SP:Clostridiales_CL:Clostridiales:Clostridiaceae:sfA |
| 39645 | Bacteria:Firmicutes:Clostridia_SP:Clostridiales_CL:Clostridiales:Clostridiaceae:sfA |
| 40015 | Bacteria:Firmicutes:Clostridia_SP:Clostridiales_CL:Clostridiales:Clostridiaceae:sfA |
| 39762 | Bacteria:Firmicutes:Clostridia_SP:Clostridiales_CL:Clostridiales:Clostridiaceae:sfA |
| 38995 | Bacteria:Firmicutes:Clostridia_SP:Clostridiales_CL:Clostridiales:Clostridiaceae:sfA |
| 40033 | Bacteria:Firmicutes:Clostridia_SP:Clostridiales_CL:Clostridiales:Clostridiaceae:sfA |
| 39297 | Bacteria:Firmicutes:Clostridia_SP:Clostridiales_CL:Clostridiales:Clostridiaceae:sfA |
| 39153 | Bacteria:Firmicutes:Clostridia_SP:Clostridiales_CL:Clostridiales:Clostridiaceae:sfA |
| 39310 | Bacteria:Firmicutes:Clostridia_SP:Clostridiales_CL:Clostridiales:Clostridiaceae:sfA |
| 39947 | Bacteria:Firmicutes:Clostridia_SP:Clostridiales_CL:Clostridiales:Clostridiaceae:sfA |
| 39741 | Bacteria:Firmicutes:Clostridia_SP:Clostridiales_CL:Clostridiales:Clostridiaceae:sfA |
| 39381 | Bacteria:Firmicutes:Clostridia_SP:Clostridiales_CL:Clostridiales:Clostridiaceae:sfA |
| 39164 | Bacteria:Firmicutes:Clostridia_SP:Clostridiales_CL:Clostridiales:Clostridiaceae:sfA |
| 39283 | Bacteria:Firmicutes:Clostridia_SP:Clostridiales_CL:Clostridiales:Clostridiaceae:sfA |
| 39682 | Bacteria:Firmicutes:Clostridia_SP:Clostridiales_CL:Clostridiales:Clostridiaceae:sfA |
| 39895 | Bacteria:Firmicutes:Clostridia_SP:Clostridiales_CL:Clostridiales:Clostridiaceae:sfA |
| 39747 | Bacteria:Firmicutes:Clostridia_SP:Clostridiales_CL:Clostridiales:Clostridiaceae:sfA |
| 39368 | Bacteria:Firmicutes:Clostridia_SP:Clostridiales_CL:Clostridiales:Clostridiaceae:sfA |
| 39237 | Bacteria:Firmicutes:Clostridia_SP:Clostridiales_CL:Clostridiales:Clostridiaceae:sfA |
| 39814 | Bacteria:Firmicutes:Clostridia_SP:Clostridiales_CL:Clostridiales:Clostridiaceae:sfA |
| 39828 | Bacteria:Firmicutes:Clostridia_SP:Clostridiales_CL:Clostridiales:Clostridiaceae:sfA |
| 39823 | Bacteria:Firmicutes:Clostridia_SP:Clostridiales_CL:Clostridiales:Clostridiaceae:sfA |
| 40020 | Bacteria:Firmicutes:Clostridia_SP:Clostridiales_CL:Clostridiales:Eubacterium_FM:sfA |
| 40029 | Bacteria:Firmicutes:Clostridia_SP:Clostridiales_CL:Clostridiales:Clostridiaceae:sfA |
| 39783 | Bacteria:Firmicutes:Clostridia_SP:Clostridiales_CL:Clostridiales:Clostridiaceae:sfA |
| 39870 | Bacteria:Firmicutes:Clostridia_SP:Clostridiales_CL:Clostridiales:Clostridiaceae:sfA |
| 39035 | Bacteria:Firmicutes:Clostridia_SP:Clostridiales_CL:Clostridiales:Clostridiaceae:sfA |
| 39763 | Bacteria:Firmicutes:Clostridia_SP:Clostridiales_CL:Clostridiales:Clostridiaceae:sfA |
| 39475 | Bacteria:Firmicutes:Clostridia_SP:Clostridiales_CL:Clostridiales:Clostridiaceae:sfA |

TABLE 5
OTUs unchanged following irradiation
Log2 Fold Change Pre vs. Post Radiation

| OTU | 0 | 4 | 11 | 21 | Log2 |
|---|---|---|---|---|---|
| 40058 | 14.35 | 13.16 | 15.08 | 14.41 | 0.13 |
| 39755 | 15.25 | 14.76 | 15.76 | 14.83 | 0.13 |
| 42581 | 15.30 | 14.31 | 15.86 | 15.36 | 0.13 |
| 3341 | 14.56 | 14.02 | 15.29 | 14.03 | 0.12 |
| 5410 | 14.79 | 14.46 | 15.26 | 14.29 | 0.12 |
| 39597 | 14.77 | 14.58 | 15.07 | 14.33 | 0.11 |
| 11139 | 15.82 | 15.74 | 16.08 | 15.34 | 0.10 |
| 39194 | 14.97 | 14.79 | 15.27 | 14.57 | 0.09 |
| 1207 | 14.38 | 13.17 | 14.81 | 14.91 | 0.09 |
| 39723 | 15.37 | 15.06 | 15.80 | 15.00 | 0.09 |
| 43035 | 14.79 | 14.50 | 15.26 | 14.37 | 0.08 |
| 39885 | 14.92 | 14.08 | 15.52 | 14.91 | 0.08 |
| 40040 | 14.78 | 14.48 | 15.26 | 14.36 | 0.08 |
| 39271 | 15.15 | 14.90 | 15.56 | 14.76 | 0.08 |
| 39852 | 14.97 | 14.80 | 15.35 | 14.53 | 0.08 |
| 39679 | 15.15 | 14.97 | 15.56 | 14.72 | 0.07 |
| 42218 | 15.05 | 14.23 | 15.86 | 14.86 | 0.06 |
| 39764 | 14.92 | 14.66 | 15.34 | 14.58 | 0.06 |
| 43443 | 14.63 | 13.86 | 15.49 | 14.36 | 0.06 |
| 42850 | 15.70 | 14.65 | 16.41 | 15.86 | 0.06 |
| 39099 | 14.95 | 14.81 | 15.24 | 14.64 | 0.05 |
| 42710 | 15.31 | 14.43 | 15.86 | 15.48 | 0.05 |
| 32681 | 15.90 | 14.58 | 16.71 | 16.26 | 0.05 |
| 40081 | 14.81 | 14.60 | 15.23 | 14.45 | 0.05 |
| 39624 | 14.86 | 14.67 | 15.30 | 14.47 | 0.05 |
| 7315 | 15.14 | 14.42 | 16.17 | 14.69 | 0.05 |
| 39269 | 15.59 | 15.39 | 15.95 | 15.30 | 0.04 |
| 3945 | 14.85 | 14.45 | 15.59 | 14.40 | 0.04 |
| 39611 | 15.62 | 15.37 | 16.03 | 15.39 | 0.03 |
| 39996 | 15.85 | 15.67 | 16.24 | 15.57 | 0.02 |
| 39013 | 15.15 | 14.85 | 15.72 | 14.82 | 0.02 |
| 39208 | 15.52 | 15.27 | 16.00 | 15.23 | 0.02 |
| 43541 | 15.89 | 15.80 | 16.17 | 15.67 | 0.01 |
| 39094 | 15.63 | 15.44 | 16.08 | 15.33 | 0.01 |
| 42884 | 15.57 | 14.72 | 16.38 | 15.60 | 0.01 |
| 1821 | 15.19 | 14.80 | 15.97 | 14.80 | 0.01 |
| 39693 | 15.18 | 15.09 | 15.61 | 14.84 | 0.00 |
| 4457 | 15.18 | 14.72 | 16.06 | 14.76 | 0.00 |
| 39139 | 15.55 | 15.43 | 16.01 | 15.25 | −0.01 |
| 241 | 14.90 | 14.19 | 15.64 | 14.90 | −0.01 |
| 43252 | 15.47 | 15.34 | 15.75 | 15.34 | −0.01 |
| 39815 | 15.16 | 14.77 | 15.80 | 14.94 | −0.01 |
| 39937 | 15.53 | 15.41 | 16.02 | 15.21 | −0.02 |
| 8943 | 14.59 | 14.17 | 15.22 | 14.46 | −0.03 |
| 5627 | 15.00 | 14.39 | 15.69 | 15.03 | −0.04 |
| 37531 | 15.31 | 14.40 | 16.21 | 15.44 | −0.04 |
| 39274 | 14.81 | 14.75 | 15.31 | 14.47 | −0.04 |
| 41940 | 15.83 | 15.25 | 16.43 | 15.93 | −0.04 |
| 39648 | 15.44 | 15.31 | 15.96 | 15.17 | −0.04 |
| 3517 | 14.46 | 14.16 | 15.37 | 13.97 | −0.04 |
| 42941 | 14.97 | 13.94 | 15.86 | 15.22 | −0.04 |
| 43911 | 14.76 | 14.51 | 15.23 | 14.67 | −0.04 |
| 42483 | 14.87 | 13.71 | 15.95 | 15.08 | −0.05 |
| 39544 | 15.00 | 14.62 | 15.71 | 14.82 | −0.05 |
| 39667 | 14.93 | 14.77 | 15.56 | 14.62 | −0.05 |
| 42965 | 15.51 | 14.67 | 16.39 | 15.63 | −0.06 |
| 43741 | 15.24 | 14.63 | 16.04 | 15.24 | −0.06 |
| 5438 | 14.71 | 14.13 | 15.46 | 14.73 | −0.06 |
| 39911 | 14.87 | 14.75 | 15.46 | 14.58 | −0.06 |
| 10036 | 15.20 | 14.70 | 15.88 | 15.21 | −0.06 |
| 39951 | 15.16 | 15.05 | 15.67 | 14.94 | −0.06 |
| 6638 | 14.22 | 13.54 | 14.93 | 14.38 | −0.06 |
| 39501 | 14.98 | 14.85 | 15.56 | 14.71 | −0.06 |
| 42924 | 15.56 | 15.14 | 16.23 | 15.49 | −0.06 |
| 42293 | 15.56 | 15.53 | 15.91 | 15.43 | −0.06 |
| 43372 | 16.01 | 15.93 | 16.30 | 15.99 | −0.06 |
| 41704 | 15.49 | 14.63 | 16.30 | 15.73 | −0.07 |
| 42604 | 15.72 | 15.09 | 16.36 | 15.92 | −0.07 |
| 39069 | 15.54 | 15.40 | 16.16 | 15.26 | −0.07 |
| 8998 | 14.34 | 13.97 | 14.88 | 14.37 | −0.07 |
| 43775 | 15.16 | 15.04 | 15.50 | 15.15 | −0.07 |
| 39875 | 14.81 | 14.47 | 15.55 | 14.62 | −0.07 |
| 6108 | 13.73 | 12.97 | 14.61 | 13.83 | −0.07 |
| 43133 | 15.02 | 14.88 | 15.51 | 14.90 | −0.08 |
| 1892 | 15.22 | 14.82 | 16.24 | 14.82 | −0.08 |
| 7499 | 15.78 | 15.73 | 16.05 | 15.80 | −0.08 |
| 17452 | 15.34 | 15.32 | 15.82 | 15.12 | −0.08 |
| 43335 | 15.59 | 15.49 | 15.96 | 15.58 | −0.08 |
| 952 | 14.74 | 13.95 | 15.81 | 14.72 | −0.08 |
| 3122 | 14.22 | 13.76 | 15.28 | 13.88 | −0.08 |
| 2202 | 15.64 | 15.03 | 16.50 | 15.66 | −0.09 |
| 4715 | 15.21 | 14.93 | 15.70 | 15.25 | −0.09 |
| 11161 | 14.88 | 14.62 | 15.34 | 14.93 | −0.09 |
| 42250 | 14.74 | 14.32 | 15.76 | 14.39 | −0.09 |
| 43391 | 15.59 | 15.53 | 15.92 | 15.57 | −0.09 |
| 43929 | 15.29 | 15.14 | 15.80 | 15.19 | −0.09 |
| 39534 | 15.19 | 14.97 | 15.85 | 15.01 | −0.09 |
| 39916 | 15.04 | 14.87 | 15.63 | 14.89 | −0.09 |
| 39707 | 15.75 | 15.72 | 16.29 | 15.53 | −0.09 |
| 39156 | 14.67 | 14.11 | 15.58 | 14.59 | −0.09 |
| 42759 | 14.60 | 14.05 | 15.38 | 14.64 | −0.09 |
| 1918 | 15.32 | 14.60 | 16.44 | 15.20 | −0.09 |
| 39414 | 14.88 | 14.74 | 15.49 | 14.70 | −0.10 |
| 28133 | 14.80 | 14.62 | 15.24 | 14.82 | −0.10 |
| 43501 | 15.83 | 15.79 | 16.17 | 15.82 | −0.10 |
| 36038 | 14.18 | 14.29 | 14.83 | 13.71 | −0.10 |
| 43610 | 15.85 | 15.77 | 16.21 | 15.85 | −0.10 |
| 3801 | 14.80 | 14.45 | 15.73 | 14.51 | −0.10 |
| 1784 | 15.11 | 14.32 | 15.82 | 15.49 | −0.10 |
| 2210 | 14.96 | 14.57 | 15.97 | 14.63 | −0.10 |
| 2307 | 15.46 | 14.70 | 16.84 | 15.15 | −0.10 |
| 43826 | 15.91 | 15.87 | 16.26 | 15.92 | −0.10 |
| 2486 | 14.53 | 14.29 | 15.33 | 14.29 | −0.10 |
| 39559 | 15.10 | 14.93 | 15.79 | 14.88 | −0.10 |
| 9590 | 14.64 | 14.23 | 15.33 | 14.66 | −0.10 |
| 278 | 14.10 | 13.33 | 15.08 | 14.21 | −0.11 |
| 41631 | 14.88 | 14.66 | 15.56 | 14.73 | −0.11 |
| 39376 | 15.45 | 15.35 | 16.07 | 15.26 | −0.11 |
| 40458 | 14.73 | 14.03 | 15.31 | 15.16 | −0.11 |
| 11384 | 14.04 | 13.31 | 14.87 | 14.26 | −0.11 |
| 43268 | 15.28 | 15.23 | 15.71 | 15.24 | −0.11 |
| 43678 | 15.80 | 15.75 | 16.22 | 15.76 | −0.11 |
| 3422 | 15.30 | 15.35 | 15.95 | 14.92 | −0.11 |
| 42934 | 15.30 | 14.86 | 16.29 | 15.09 | −0.11 |
| 38464 | 14.74 | 13.78 | 15.26 | 15.50 | −0.11 |
| 39516 | 15.30 | 14.96 | 16.01 | 15.25 | −0.11 |
| 43544 | 15.30 | 15.15 | 15.56 | 15.54 | −0.11 |
| 4764 | 14.40 | 13.94 | 15.38 | 14.21 | −0.11 |
| 43658 | 16.04 | 16.02 | 16.42 | 16.03 | −0.12 |
| 5475 | 14.60 | 14.03 | 15.46 | 14.66 | −0.12 |
| 39223 | 15.33 | 15.14 | 15.97 | 15.24 | −0.12 |
| 11663 | 15.28 | 15.18 | 15.95 | 15.06 | −0.12 |
| 42595 | 15.20 | 14.53 | 16.03 | 15.39 | −0.12 |
| 110 | 14.59 | 14.34 | 15.35 | 14.44 | −0.12 |
| 3560 | 14.78 | 14.64 | 15.52 | 14.54 | −0.12 |
| 43819 | 15.30 | 15.21 | 15.77 | 15.29 | −0.12 |
| 42328 | 14.54 | 13.65 | 15.49 | 14.86 | −0.12 |
| 4217 | 15.35 | 15.22 | 16.11 | 15.09 | −0.12 |
| 4595 | 15.12 | 14.90 | 15.81 | 15.02 | −0.12 |
| 42569 | 15.01 | 14.01 | 16.01 | 15.39 | −0.12 |
| 40518 | 14.90 | 14.08 | 15.84 | 15.16 | −0.12 |
| 43280 | 15.79 | 15.72 | 16.21 | 15.81 | −0.12 |
| 650 | 14.68 | 14.30 | 15.54 | 14.59 | −0.13 |
| 43539 | 15.18 | 15.15 | 15.53 | 15.23 | −0.13 |
| 43349 | 15.64 | 15.53 | 16.16 | 15.62 | −0.13 |
| 43488 | 16.07 | 16.06 | 16.44 | 16.10 | −0.13 |
| 43270 | 15.10 | 14.81 | 15.73 | 15.16 | −0.13 |
| 43334 | 15.00 | 14.87 | 15.62 | 14.93 | −0.13 |
| 39515 | 15.86 | 15.82 | 16.44 | 15.72 | −0.13 |
| 36985 | 15.47 | 15.24 | 16.33 | 15.24 | −0.14 |
| 4229 | 14.60 | 14.28 | 15.58 | 14.34 | −0.14 |
| 43795 | 15.66 | 15.61 | 16.16 | 15.63 | −0.14 |

TABLE 6
Taxonomy corresponding to OTUs unchanged following irradiation

| OTU | Full Taxonomy |
|---|---|
| 40058 | Bacteria:Firmicutes:Clostridia_SP:Clostridiales_CL:Clostridiales:Clostridiaceae:sfA |
| 39755 | Bacteria:Firmicutes:Clostridia_SP:Clostridiales_CL:Clostridiales:Clostridiaceae:sfA |
| 42581 | Bacteria:Firmicutes:Clostridia_SP:RF30_CL:Clostridiales:RF6_FM:sfA |
| 3341 | Bacteria:Firmicutes:Clostridia_SP:C23_c19_CL:Clostridiales:Unclassified:sfA |
| 5410 | Bacteria:Firmicutes:Clostridia_SP:Clostridiales_CL:Clostridiales:Ruminococcus_FM:sfA |
| 39597 | Bacteria:Firmicutes:Clostridia_SP:Clostridiales_CL:Clostridiales:Clostridiaceae:sfA |
| 11139 | Bacteria:Firmicutes:Bacilli_SP:Lactobacillales_CL:Lactobacillales:Aerococcaceae:sfA |
| 39194 | Bacteria:Firmicutes:Clostridia_SP:Clostridiales_CL:Clostridiales:Clostridiaceae:sfA |
| 1207 | Bacteria:Firmicutes:Clostridia_SP:Clostridiales_CL:Clostridiales:Clostridium_FM:sfA |
| 39723 | Bacteria:Firmicutes:Clostridia_SP:Clostridiales_CL:Clostridiales:Clostridiaceae:sfA |
| 43035 | Bacteria:Firmicutes:Clostridia_SP:Clostridiales_CL:Clostridiales:Clostridiaceae:sfA |
| 39885 | Bacteria:Firmicutes:Clostridia_SP:Clostridiales_CL:Clostridiales:Clostridiaceae:sfA |
| 40040 | Bacteria:Firmicutes:Clostridia_SP:Clostridiales_CL:Clostridiales:Clostridiaceae:sfA |
| 39271 | Bacteria:Firmicutes:Clostridia_SP:Clostridiales_CL:Clostridiales:Clostridiaceae:sfA |
| 39852 | Bacteria:Firmicutes:Clostridia_SP:Clostridiales_CL:Clostridiales:Clostridiaceae:sfA |
| 39679 | Bacteria:Firmicutes:Clostridia_SP:Clostridiales_CL:Clostridiales:Clostridiaceae:sfA |
| 42218 | Bacteria:Firmicutes:Clostridia_SP:RF30_CL:Clostridiales:RF6_FM:sfA |
| 39764 | Bacteria:Firmicutes:Clostridia_SP:Clostridiales_CL:Clostridiales:Clostridiaceae:sfA |
| 43443 | Bacteria:Firmicutes:Clostridia_SP:RL197_aah88c10_CL:Clostridiales:Unclassified:sfA |
| 42850 | Bacteria:Firmicutes:Clostridia_SP:RF30_CL:Clostridiales:RF6_FM:sfA |
| 39099 | Bacteria:Firmicutes:Clostridia_SP:Clostridiales_CL:Clostridiales:Clostridiaceae:sfA |
| 42710 | Bacteria:Firmicutes:Clostridia_SP:RF30_CL:Clostridiales:RF6_FM:sfA |
| 32681 | Bacteria:Firmicutes:Clostridia_SP:Catabacter_CL:Catabacter_OR:Catabacter_FM:sfL |
| 40081 | Bacteria:Firmicutes:Clostridia_SP:Clostridiales_CL:Clostridiales:Clostridiaceae:sfA |
| 39624 | Bacteria:Firmicutes:Clostridia_SP:Clostridiales_CL:Clostridiales:Clostridiaceae:sfA |
| 7315 | Bacteria:Firmicutes:Clostridia_SP:Clostridiales_CL:Clostridiales:Unclassified:sfA |
| 39269 | Bacteria:Firmicutes:Clostridia_SP:Clostridiales_CL:Clostridiales:Clostridiaceae:sfA |
| 3945 | Bacteria:Firmicutes:Clostridia_SP:C23_c19_CL:Clostridiales:Unclassified:sfA |
| 39611 | Bacteria:Firmicutes:Clostridia_SP:Clostridiales_CL:Clostridiales:Clostridiaceae:sfA |
| 39996 | Bacteria:Firmicutes:Clostridia_SP:Clostridiales_CL:Clostridiales:Clostridiaceae:sfA |
| 39013 | Bacteria:Firmicutes:Clostridia_SP:Clostridiales_CL:Clostridiales:Clostridiaceae:sfA |
| 39208 | Bacteria:Firmicutes:Clostridia_SP:Clostridiales_CL:Clostridiales:Clostridiaceae:sfA |
| 43541 | Bacteria:Firmicutes:Clostridia_SP:Clostridiales_CL:Clostridiales:Clostridiaceae:sfA |
| 39094 | Bacteria:Firmicutes:Clostridia_SP:Clostridiales_CL:Clostridiales:Clostridiaceae:sfA |
| 42884 | Bacteria:Firmicutes:Clostridia_SP:RF30_CL:Clostridiales:RF6_FM:sfA |
| 1821 | Bacteria:Firmicutes:Clostridia_SP:BB68_CL:Clostridiales:Unclassified:sfA |
| 39693 | Bacteria:Firmicutes:Clostridia_SP:Clostridiales_CL:Clostridiales:Clostridiaceae:sfA |
| 4457 | Bacteria:Firmicutes:Clostridia_SP:Clostridiales_CL:Clostridiales:Clostridium_FM:sfA |
| 39139 | Bacteria:Firmicutes:Clostridia_SP:Clostridiales_CL:Clostridiales:Clostridiaceae:sfA |
| 241 | Bacteria:Firmicutes:Clostridia_SP:Clostridiales_CL:Clostridiales:Unclassified:sfA |
| 43252 | Bacteria:Firmicutes:Clostridia_SP:Peptostreptococcaceae_CL:Peptostreptococcaceae_OR:Peptostreptococcaceae:sfA |
| 39815 | Bacteria:Firmicutes:Clostridia_SP:Clostridiales_CL:Clostridiales:Clostridiaceae:sfA |
| 39937 | Bacteria:Firmicutes:Clostridia_SP:Clostridiales_CL:Clostridiales:Clostridiaceae:sfA |
| 8943 | Bacteria:Firmicutes:Bacilli_SP:Lactobacillales_CL:Lactobacillales:Leuconostoc_FM:sfA |
| 5627 | Bacteria:Firmicutes:Clostridia_SP:Clostridiales_CL:Clostridiales:Unclassified:sfA |
| 37531 | Bacteria:Firmicutes:Clostridia_SP:SR5_CL:Clostridiales:Unclassified:sfG |
| 39274 | Bacteria:Firmicutes:Clostridia_SP:Clostridiales_CL:Clostridiales:Clostridiaceae:sfA |
| 41940 | Bacteria:Firmicutes:Clostridia_SP:RF30_CL:Clostridiales:RF6_FM:sfA |
| 39648 | Bacteria:Firmicutes:Clostridia_SP:Clostridiales_CL:Clostridiales:Clostridiaceae:sfA |
| 3517 | Bacteria:Firmicutes:Clostridia_SP:C23_c19_CL:Clostridiales:Unclassified:sfA |
| 42941 | Bacteria:Firmicutes:Clostridia_SP:RF30_CL:Clostridiales:RF6_FM:sfA |
| 43911 | Bacteria:Firmicutes:Clostridia_SP:Peptostreptococcaceae_CL:Peptostreptococcaceae_OR:Peptostreptococcaceae:sfA |
| 42483 | Bacteria:Firmicutes:Clostridia_SP:RF30_CL:Clostridiales:RF6_FM:sfA |
| 39544 | Bacteria:Firmicutes:Clostridia_SP:Clostridiales_CL:Clostridiales:Clostridiaceae:sfA |
| 39667 | Bacteria:Firmicutes:Clostridia_SP:Clostridiales_CL:Clostridiales:Clostridiaceae:sfA |
| 42965 | Bacteria:Firmicutes:Clostridia_SP:RF30_CL:Clostridiales:RF6_FM:sfA |
| 43741 | Bacteria:Firmicutes:Clostridia_SP:RL197_aah88c10_CL:Clostridiales:Unclassified:sfA |
| 5438 | Bacteria:Firmicutes:Clostridia_SP:Clostridiales_CL:Clostridiales:Unclassified:sfA |
| 39911 | Bacteria:Firmicutes:Clostridia_SP:Clostridiales_CL:Clostridiales:Clostridiaceae:sfA |
| 10036 | Bacteria:Firmicutes:Bacilli_SP:Lactobacillales_CL:Lactobacillales:Streptococcaceae:sfA |
| 39951 | Bacteria:Firmicutes:Clostridia_SP:Clostridiales_CL:Clostridiales:Clostridiaceae:sfA |
| 6638 | Bacteria:Firmicutes:Clostridia_SP:Clostridiales_CL:Clostridiales:Unclassified:sfA |
| 39501 | Bacteria:Firmicutes:Clostridia_SP:Clostridiales_CL:Clostridiales:Clostridiaceae:sfA |
| 42924 | Bacteria:Firmicutes:Clostridia_SP:RF30_CL:Clostridiales:RF6_FM:sfA |
| 42293 | Bacteria:Firmicutes:Clostridia_SP:Peptostreptococcaceae_CL:Peptostreptococcaceae_OR:Peptostreptococcaceae:sfA |
| 43372 | Bacteria:Firmicutes:Clostridia_SP:Peptostreptococcaceae_CL:Peptostreptococcaceae_OR:Peptostreptococcaceae:sfA |
| 41704 | Bacteria:Firmicutes:Mollicutes_SP:RF39_CL:Unclassified:Unclassified:sfA |
| 42604 | Bacteria:Firmicutes:Clostridia_SP:RF30_CL:Clostridiales:RF6_FM:sfA |
| 39069 | Bacteria:Firmicutes:Clostridia_SP:Clostridiales_CL:Clostridiales:Clostridiaceae:sfA |
| 8998 | Bacteria:Firmicutes:Bacilli_SP:Lactobacillales_CL:Lactobacillales:Leuconostoc_FM:sfA |
| 43775 | Bacteria:Firmicutes:Clostridia_SP:Peptostreptococcaceae_CL:Peptostreptococcaceae_OR:Peptostreptococcaceae:sfA |
| 39875 | Bacteria:Firmicutes:Clostridia_SP:Clostridiales_CL:Clostridiales:Clostridiaceae:sfA |
| 6108 | Bacteria:Firmicutes:Clostridia_SP:Clostridiales_CL:Clostridiales:Clostridium_bolteae:sfA |
| 43133 | Bacteria:Firmicutes:Clostridia_SP:Peptostreptococcaceae_CL:Peptostreptococcaceae_OR:Peptostreptococcaceae:sfA |
| 1892 | Bacteria:Firmicutes:Clostridia_SP:Clostridiales_CL:Clostridiales:Unclassified:sfA |
| 7499 | Bacteria:Firmicutes:Clostridia_SP:Peptostreptococcaceae_CL:Peptostreptococcaceae_OR:Peptostreptococcaceae:sfA |
| 17452 | Bacteria:Firmicutes:Gelria_SP:Gelria_CL:Gelria_OR:Gelria_FM:sfA |
| 43335 | Bacteria:Firmicutes:Clostridia_SP:Peptostreptococcaceae_CL:Peptostreptococcaceae_OR:Peptostreptococcaceae:sfA |
| 952 | Bacteria:Firmicutes:Clostridia_SP:Clostridiales_CL:Clostridiales:Coprococcus_FM:sfA |
| 3122 | Bacteria:Firmicutes:Clostridia_SP:C23_k02_CL:Clostridiales:C22_o06_FM:sfA |
| 2202 | Bacteria:Firmicutes:Clostridia_SP:Clostridiales_CL:Clostridiales:Clostridium_bolteae:sfA |
| 4715 | Bacteria:Firmicutes:Clostridia_SP:Clostridiales_CL:Clostridiales:Ruminococcus_FM:sfA |
| 11161 | Bacteria:Firmicutes:Bacilli_SP:Lactobacillales_CL:Lactobacillales:Aerococcaceae:sfA |
| 42250 | Bacteria:Firmicutes:Clostridia_SP:Unclassified:Unclassified:Unclassified:sfA |
| 43391 | Bacteria:Firmicutes:Clostridia_SP:Peptostreptococcaceae_CL:Peptostreptococcaceae_OR:Peptostreptococcaceae:sfA |
| 43929 | Bacteria:Firmicutes:Clostridia_SP:Peptostreptococcaceae_CL:Peptostreptococcaceae_OR:Peptostreptococcaceae:sfA |
| 39534 | Bacteria:Firmicutes:Clostridia_SP:Clostridiales_CL:Clostridiales:Clostridiaceae:sfA |
| 39916 | Bacteria:Firmicutes:Clostridia_SP:Clostridiales_CL:Clostridiales:Clostridiaceae:sfA |
| 39707 | Bacteria:Firmicutes:Clostridia_SP:Clostridiales_CL:Clostridiales:Clostridiaceae:sfA |
| 39156 | Bacteria:Firmicutes:Clostridia_SP:Clostridiales_CL:Clostridiales:Clostridiaceae:sfA |
| 42759 | Bacteria:Firmicutes:Mollicutes_SP:Catenibacterium_CL:Catenibacterium_OR:Catenibacterium_FM:sfA |
| 1918 | Bacteria:Firmicutes:Clostridia_SP:Clostridiales_CL:Clostridiales:Eubacterium_FM:sfA |
| 39414 | Bacteria:Firmicutes:Clostridia_SP:Clostridiales_CL:Clostridiales:Clostridiaceae:sfA |
| 28133 | Bacteria:Firmicutes:Clostridia_SP:Peptostreptococcaceae_CL:Peptostreptococcaceae_OR:Peptostreptococcaceae:sfA |
| 43501 | Bacteria:Firmicutes:Clostridia_SP:Peptostreptococcaceae_CL:Peptostreptococcaceae_OR:Peptostreptococcaceae:sfA |
| 36038 | Bacteria:Firmicutes:Clostridia_SP:butyrate-producing_bacterium_A2-207_CL:Clostridiales:Unclassified:sfA |
| 43610 | Bacteria:Firmicutes:Clostridia_SP:Peptostreptococcaceae_CL:Peptostreptococcaceae_OR:Peptostreptococcaceae:sfA |
| 3801 | Bacteria:Firmicutes:Clostridia_SP:C23_c19_CL:Clostridiales:Unclassified:sfA |
| 1784 | Bacteria:Firmicutes:Clostridia_SP:Clostridiales_CL:Clostridiales:Clostridium_FM:sfA |
| 2210 | Bacteria:Firmicutes:Clostridia_SP:C23_k02_CL:Clostridiales:C22_o06_FM:sfA |
| 2307 | Bacteria:Firmicutes:Clostridia_SP:Clostridiales_CL:Clostridiales:Unclassified:sfA |
| 43826 | Bacteria:Firmicutes:Clostridia_SP:Peptostreptococcaceae_CL:Peptostreptococcaceae_OR:Peptostreptococcaceae:sfA |
| 2486 | Bacteria:Firmicutes:Clostridia_SP:Clostridiales_CL:Clostridiales:Clostridium_FM:sfA |
| 39559 | Bacteria:Firmicutes:Clostridia_SP:Clostridiales_CL:Clostridiales:Clostridiaceae:sfA |
| 9590 | Bacteria:Firmicutes:Bacilli_SP:Lactobacillales_CL:Lactobacillales:Leuconostoc_FM:sfA |
| 278 | Bacteria:Firmicutes:Clostridia_SP:Clostridiales_CL:Clostridiales:Eubacterium_FM:sfA |
| 41631 | Bacteria:Firmicutes:Clostridia_SP:Clostridiales_CL:Clostridiales:Eubacterium_FM:sfO |
| 39376 | Bacteria:Firmicutes:Clostridia_SP:Clostridiales_CL:Clostridiales:Clostridiaceae:sfA |
| 40458 | Bacteria:Firmicutes:Mollicutes_SP:RF39_CL:p-3870-23G5_OR:Unclassified:sfD |
| 11384 | Bacteria:Firmicutes:Bacilli_SP:Lactobacillales_CL:Lactobacillales:Lactobacillaceae:sfA |
| 43268 | Bacteria:Firmicutes:Clostridia_SP:Peptostreptococcaceae_CL:Peptostreptococcaceae_OR:Peptostreptococcaceae:sfA |
| 43678 | Bacteria:Firmicutes:Clostridia_SP:Peptostreptococcaceae_CL:Peptostreptococcaceae_OR:Peptostreptococcaceae:sfA |
| 3422 | Bacteria:Firmicutes:Clostridia_SP:BB68_CL:Clostridiales:Unclassified:sfA |
| 42934 | Bacteria:Firmicutes:Clostridia_SP:Unclassified:Unclassified:Unclassified:sfA |
| 38464 | Bacteria:Firmicutes:Clostridia_SP:p-4154-6Wa5_CL:Clostridiales:C10_I02_FM:sfA |
| 39516 | Bacteria:Firmicutes:Clostridia_SP:Clostridiales_CL:Clostridiales:Clostridiaceae:sfA |
| 43544 | Bacteria:Firmicutes:Clostridia_SP:Peptostreptococcaceae_CL:Peptostreptococcaceae_OR:Peptostreptococcaceae:sfA |
| 4764 | Bacteria:Firmicutes:Clostridia_SP:Clostridiales_CL:Clostridiales:Ruminococcus_FM:sfA |
| 43658 | Bacteria:Firmicutes:Clostridia_SP:Peptostreptococcaceae_CL:Peptostreptococcaceae_OR:Peptostreptococcaceae:sfA |
| 5475 | Bacteria:Firmicutes:Clostridia_SP:Clostridiales_CL:Clostridiales:Ruminococcus_FM:sfA |
| 39223 | Bacteria:Firmicutes:Clostridia_SP:Clostridiales_CL:Clostridiales:Clostridiaceae:sfA |
| 11663 | Bacteria:Firmicutes:Bacilli_SP:Lactobacillales_CL:Lactobacillales:Aerococcaceae:sfA |
| 42595 | Bacteria:Firmicutes:Mollicutes_SP:Catenibacterium_CL:Catenibacterium_OR:Catenibacterium_FM:sfA |
| 110 | Bacteria:Firmicutes:Clostridia_SP:Clostridiales_CL:Clostridiales:Ruminococcus_FM:sfA |
| 3560 | Bacteria:Firmicutes:Clostridia_SP:Clostridiales_CL:Clostridiales:Unclassified:sfA |
| 43819 | Bacteria:Firmicutes:Clostridia_SP:Peptostreptococcaceae_CL:Peptostreptococcaceae_OR:Peptostreptococcaceae:sfA |
| 42328 | Bacteria:Firmicutes:Clostridia_SP:RF30_CL:Clostridiales:RF6_FM:sfA |
| 4217 | Bacteria:Firmicutes:Clostridia_SP:Clostridiales_CL:Clostridiales:Ruminococcus_FM:sfA |
| 4595 | Bacteria:Firmicutes:Clostridia_SP:Clostridiales_CL:Clostridiales:Johnsonella_FM:sfA |
| 42569 | Bacteria:Firmicutes:Clostridia_SP:RF30_CL:Clostridiales:RF6_FM:sfA |
| 40518 | Bacteria:Firmicutes:Clostridia_SP:Peptostreptococcaceae_CL:Peptostreptococcaceae_OR:Peptostreptococcaceae:sfA |
| 43280 | Bacteria:Firmicutes:Clostridia_SP:Peptostreptococcaceae_CL:Peptostreptococcaceae_OR:Peptostreptococcaceae:sfA |
| 650 | Bacteria:Firmicutes:Clostridia_SP:Clostridiales_CL:Clostridiales:Eubacterium_FM:sfA |
| 43539 | Bacteria:Firmicutes:Clostridia_SP:Peptostreptococcaceae_CL:Peptostreptococcaceae_OR:Peptostreptococcaceae:sfA |
| 43349 | Bacteria:Firmicutes:Clostridia_SP:Peptostreptococcaceae_CL:Peptostreptococcaceae_OR:Peptostreptococcaceae:sfA |
| 43488 | Bacteria:Firmicutes:Clostridia_SP:Peptostreptococcaceae_CL:Peptostreptococcaceae_OR:Peptostreptococcaceae:sfA |
| 43270 | Bacteria:Firmicutes:Clostridia_SP:Clostridiales_CL:Clostridiales:Ruminococcus_FM:sfA |
| 43334 | Bacteria:Firmicutes:Clostridia_SP:Peptostreptococcaceae_CL:Peptostreptococcaceae_OR:Peptostreptococcaceae:sfA |
| 39515 | Bacteria:Firmicutes:Clostridia_SP:Clostridiales_CL:Clostridiales:Clostridiaceae:sfA |
| 36985 | Bacteria:Firmicutes:Clostridia_SP:p-3024-SwA5_CL:Clostridiales:Unclassified:sfA |
| 4229 | Bacteria:Firmicutes:Clostridia_SP:adhufec25_CL:Clostridiales:Unclassified:sfA |
| 43795 | Bacteria:Firmicutes:Clostridia_SP:Peptostreptococcaceae_CL:Peptostreptococcaceae_OR:Peptostreptococcaceae:sfA |

1. A method of identifying and/or characterizing exposure to radiation comprising:
providing a reference microbial signature that correlates with one or more features of exposure to radiation; and
determining a microbial signature present in a microbiota sample from an individual whose exposure to radiation is to be identified or characterized.

2. The method of claim 1 wherein a microbiota sample comprises a sample of one or more types of microbes found in a particular organ or tissue of an individual.

3. The method of claim 1 wherein a microbiota sample comprises a sample of substantially all types of microbes found in a particular organ or tissue of an individual.

4. The method of claim 2 wherein the particular organ or tissue of an individual is a gastrointestinal tract of an individual.

5. The method of claim 4 wherein the microbiota sample is a fecal sample.

6. The method of claim 1 wherein the microbial signature comprises a level or set of levels of one or more types of microbes or components or products thereof present in a microbial sample.

7. The method of claim 1 wherein the microbial signature comprises a set of levels of a set of substantially all types of microbes or components or products thereof present in a microbial sample.

8. The method of claim 1 wherein the microbial signature comprises an activity level of one or more types of microbes or components or products thereof present in microbial sample.

9. The method of claim 6 wherein the microbial signature comprises a level or set of levels of one or more DNA sequences.

10. The method of claim 6 wherein the microbial signature comprises a level or set of levels of one or more 16S rRNA gene sequences.

11. The method of claim 10 wherein at least one of the 16S rRNA gene sequences are selected from clostridium, sarcina, barnesiella, lactobacillus, or streptococcus.

12. The method of claim 10 wherein at least one of the 16S rRNA gene sequences is from firmicutes.

13. The method of claim 1 wherein the individual is human.

14. The method of claim 1 wherein the individual is an animal.

15. The method of claim 1 wherein an individual is a plant.

16. The method of claim 1 wherein a feature of radiation exposure comprises a dose of radiation exposure.

17. The method of claim 1 wherein a feature of radiation exposure comprises a type of radiation exposure.

18. A method of defining a microbial signature that correlates with an aspect of radiation exposure, the method comprising steps of:
determining a first set of levels of one or more types of microbes or components or products thereof in a first collection of microbiota samples, where each sample in the first collection of microbiota samples shares a common feature of radiation exposure;
determining a second set of levels of the one or more types of microbes or components or products thereof in a second collection of microbiota samples, which second collection of microbiota samples does not share the common feature of radiation exposure but is otherwise comparable to the first set of microbiota samples;
identifying a microbial signature comprising levels within the first or second set that correlates with presence or absence of the common feature of radiation exposure.

19. The method of claim 18 wherein microbiota samples comprise samples of one or more types of microbes found in particular organs or tissues from which the microbiota samples are collected.

20. The method of claim 18 wherein microbiota samples comprises samples of substantially all types of microbes found in particular organs or tissues from which the microbiota samples are collected.

21. The method of claim 19 wherein the particular organ or tissue is a gastrointestinal tract.

22. The method of claim 18, wherein the common feature of radiation exposure comprises a duration of exposure.

23. The method of claim 18, wherein the common feature of radiation exposure comprises a duration of time post-exposure.

24. The method of claim 18, wherein the common feature of radiation exposure comprises a dose of exposure.

25. The method of claim 24, wherein the dose of radiation exposure is from 0 to 10 Gy.

26. The method of claim 18, wherein the common feature of radiation exposure comprises a type of exposure.

27. The method of claim 18 wherein a set of levels of one or more types of microbes or components or products thereof comprises a set of levels of DNA sequences of one or more types of microbes.

28. The method of claim 27 wherein a set of levels of one or more types of microbes or components or products thereof comprises a set of levels of 16S rRNA gene sequences of one or more types of microbes.


SEQUENCE LISTING (14 sequences)

SEQ ID NO: 1 - DNA, 19 bp, Artificial Sequence
  BactF285F Primer Sequence
ggttctgagaggaggtccc

SEQ ID NO: 2 - DNA, 18 bp, Artificial Sequence
  UniR338 Primer Sequence
gctgcctcccgtaggagt

SEQ ID NO: 3 - DNA, 18 bp, Artificial Sequence
  UniF338 Primer Sequence
actcctacgggaggcagc

SEQ ID NO: 4 - DNA, 23 bp, Artificial Sequence
  CcocR491 Primer Sequence
gcttcttagtcaggtaccgtcat

SEQ ID NO: 5 - DNA, 19 bp, Artificial Sequence
  Uni515F Primer Sequence
gtgccagcmgccgcggtaa

SEQ ID NO: 6 - DNA, 23 bp, Artificial Sequence
  Ent826R Primer Sequence
gcctcaagggcacaacctccaag

SEQ ID NO: 7 - DNA, 22 bp, Artificial Sequence
  StableF Primer Sequence
ttcgcttctcttcgtatgcggc

SEQ ID NO: 8 - DNA, 21 bp, Artificial Sequence
  StableR Primer Sequence
tcttcacacacgcggcatggc

SEQ ID NO: 9 - DNA, 20 bp, Artificial Sequence
  DownF Primer Sequence
cgcgtgggtaacctgccctg

SEQ ID NO: 10 - DNA, 22 bp, Artificial Sequence
  DownR Primer Sequence
cgcgggtccatcctataccgca

SEQ ID NO: 11 - DNA, 20 bp, Artificial Sequence
  AcuteF Primer Sequence
tcgggcctcttgccatcgga

SEQ ID NO: 12 - DNA, 21 bp, Artificial Sequence
  AcuteR Primer Sequence
ccggttaacgcttgcacccct

SEQ ID NO: 13 - DNA, 20 bp, Artificial Sequence
  ChronicF Primer Sequence
ctgggatggacctgcggtgt

SEQ ID NO: 14 - DNA, 24 bp, Artificial Sequence
  ChronicR Primer Sequence
ttacgagccgaaacccttcttcac